(19) United States
(12) Patent Application Publication
(10) Pub. No.: US 2026/0174741 A1
(43) Pub. Date: Jun. 25, 2026

(54) METHODS OF REDUCING MIRO1 OR PHOSPHORYLATED ALPHA-SYNUCLEIN

(71) Applicant: Acurex Biosciences Corporation, San Carlos, CA (US)

(72) Inventors: Sean PINTCHOVSKI, San Carlos, CA (US); William D. SHRADER, San Carlos, CA (US)

(21) Appl. No.: 19/125,645

(22) PCT Filed: Oct. 30, 2023
(86) PCT No.: PCT/US2023/078151

§ 371 Date: Apr. 29, 2025

Related U.S. Application Data
(60) Provisional application No. 63/381,662, filed on Oct. 31, 2022.

(51) Int. Cl. A61K 31/437; A61K 31/5415; A61P 25/16

(52) U.S. Cl. CPC A61K 31/437; A61K 31/5415; A61P 25/16

(57) ABSTRACT

The present disclosure describes a method of reducing Miro1 and/or phosphorylated alpha-synuclein level in a cell, for example, by contacting the cell with an effective amount of a T-type calcium channel antagonist or a ferroptosis inhibitor.

### CROSS-REFERENCES TO RELATED APPLICATIONS

This application is a claims priority to U.S. provisional appl. No. 63/381,662, filed Oct. 31, 2022, which is incorporated by reference herein in its entirety for all purposes.

### BACKGROUND OF THE INVENTION

Alpha-synuclein (α-Syn) is a major constituent of Lewy bodies and a pathogenic hallmark of all synucleinopathies, including Parkinson's disease (PD), dementia with Lewy bodies (DLB), and multiple system atrophy (MSA). α-Synuclein is linked to the pathogenesis of both familial and sporadic forms of Parkinson's disease. Several posttranslational modifications to α-synuclein are known to occur in PD. Among them is phosphorylation at Ser¹²⁹ [phosphorylated α-synuclein (P-S129)], a modification that may be critical in PD pathogenesis. P-S129 has been reported to enhance α-synuclein toxicity both in vivo and in vitro. Since the P-S129 modification of α-synuclein contributes to PD pathology and is found at high concentrations in the key pathological hallmarks of PD, Lewy Bodies and Lewy Neurites, a novel method to reduce levels of P-S129 α-synuclein may provide therapeutic benefit to individuals suffering from PD.

Miro1 is an outer mitochondrial membrane (OMM) protein that anchors the microtubule motors kinesin and dynein to mitochondria. Altered mitochondrial transport is one of the pathogenic changes in major adult-onset neurodegenerative diseases. In both sporadic and familial mutant LRRK2 GS2019, SNCA A53T, and GBA N370S cells derived from PD subjects, the mitochondrial outer membrane protein Miro1 is stabilized and remains on damaged mitochondria for longer than normal, prolonging active transport and inhibiting mitochondrial degradation. Miro1 degradation and mitochondrial motility are also impaired in sporadic PD patients. Prolonged retention of Miro1, and the downstream consequences that ensue, may constitute a central component of PD pathogenesis. Since Miro1 levels are found at high concentrations in the key pathological hallmarks of PD, a novel method to reduce levels of P-S129 α-synuclein may provide therapeutic benefit to individuals suffering from PD.

T-type calcium channel antagonists have been reported to be able to reduce Miro1 levels in a Parkinson's disease fibroblast assay. See, WO 2022/216386, page 203, Table 6.

There is a need for methods of reducing Miro1 and/or phosphorylated alpha-synuclein, for example, in a method of treating Parkinson's disease.

### BRIEF SUMMARY OF THE INVENTION

In some embodiments, a method of the present disclosure is a method of reducing Miro1 and/or phosphorylated alpha-synuclein level in a cell, comprising contacting the cell with an effective amount of a compound having Formula I:

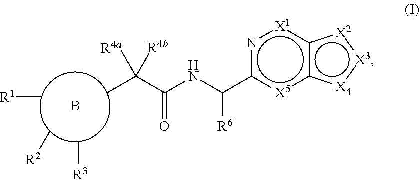

or a pharmaceutically acceptable salt thereof,
wherein
ring B is C₆-C₁₀ aryl or 5- to 10-membered heteroaryl;
R¹, R², and R³ are each independently H, halogen, CN, OR¹¹, NR^{12a}R^{12b}, C₁-C₆ alkyl, C₂-C₆ alkoxyalkyl, C₁-C₆ haloalkyl, C₂-C₆ alkenyl, C₂-C₆ alkynyl, phenyl, 5- to 6-membered heteroaryl, C₃-C₇ cycloalkyl, —(CH₂)ₘ—(C₃-C₇ cycloalkyl), 4- to 7-membered heterocyclyl, or —(CH₂)ₘ-(4- to 7-membered heterocyclyl), wherein the phenyl, heteroaryl, cycloalkyl or heterocyclyl is optionally substituted by 1, 2, or 3 halogen, CN, OR¹¹, NR^{12a}R^{12b}, C₁-C₆ alkyl, C₁-C₆ alkoxy, C₁-C₆ alkylamino, or C₁-C₆ haloalkyl;
R^{4a} and R^{4b} are each independently H or C₁-C₆ alkyl, or R^{4a} and R^{4b} and the carbon atom to which they are attached form a C₃-C₅ cycloalkyl;
R⁶ is H, C₁-C₆ alkyl, or C₁-C₆ haloalkyl;
X¹ and X⁵ are each independently N or CR⁸, provided that at least one of X¹ and X⁵ is CR⁸;
X², X³, and X⁴ are each independently N, NR⁹, O, S, or CR⁹, provided that at least one of X², X³, and X⁴ is N, NR⁹, O, or S;
R⁸ is H, halogen, CN, OR¹¹, C₁-C₆ alkyl, C₂-C₆ alkoxyalkyl, or C₁-C₆ haloalkyl;
R⁹ is H, C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen, CN, OH, NH₂, C₁-C₆ alkyl, C₁-C₆ alkoxy, C₁-C₆alkylamino, or C₁-C₆ haloalkyl;
each R¹¹, R^{12a}, and R^{12b} is independently H, C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen or CN; and
subscript m is 1, 2, or 3;
or a compound having Formula II:

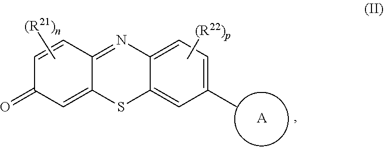

or a pharmaceutically acceptable salt thereof,
wherein
each R²¹ is independently —F, —Cl, —Br, —I, —OR^{a}, —SR^{a}, —NR^{a}R^{b}, —NO₂, —CN, C₁₋₆ alkyl, C₂₋₆ alkenyl, or C₂₋₆ alkynyl, wherein the alkyl, alkenyl, or alkynyl is substituted with 0, 1, 2, or 3 groups independently selected from —F, —Cl, —Br, —I, —OR^{a}, —SR^{a}, —NR^{a}R^{b}, oxo, —NO₂, and —CN; each R²² is independently —F, —Cl, —Br, —I, —OH, —OR^{a}, —SR^{a}, —NR^{a}R^{b}, —CN, C₁₋₆ alkyl, C₂₋₆ alkenyl, or C₂₋₆ alkynyl, wherein the alkyl, alkenyl, or alkynyl is substituted with 0, 1, 2, or 3 groups independently selected from —F, —Cl, —Br, —I, —OR^{a}, —SR^{a}, —NR^{a}R^{b}, oxo, —NO₂, and —CN;
each R^{a} is independently H or C₁₋₆ alkyl;
each R^{b} is independently H or C₁₋₆ alkyl;
the subscript n is 0, 1, 2 or 3;
the subscript p is 0, 1, 2, or 3; and

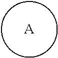

is a nitrogen-containing heterocyclic ring.

In some embodiments, a method of the present disclosure is a method of treating a disease or condition characterized by an elevated Miro1 and/or phosphorylated alpha-synuclein level in a subject in need thereof, comprising administering to the subject a therapeutically effective amount of a compound having Formula I or Formula II, or a pharmaceutically acceptable salt thereof.

### BRIEF DESCRIPTION OF THE DRAWINGS

FIG. 1A-1C show effects in rat primary midbrain neuron co-cultures of alpha-synuclein oligomer (“α-Syn”) challenge alone, in combination with Compound 1 (“α-Syn+Compound 1”) or in combination with positive control (“α-Syn+Pos. Control”) as compared to control (“Control”) as measured in tyrosine-hydroxylase positive (TH+) neuron count (FIG. 1A), Miro1 levels (FIG. 1B), or phosphorylated alpha-synuclein (FIG. 1C). Bars show mean±SEM. One way ANOVA with post hoc Dunnett's t-test: **** (p<0.0001), *** (p<0.001), ** (p<0.01), * (p<0.05), ns (not significant).

FIG. 2A-2B show effects in rat primary midbrain neuron co-cultures of alpha-synuclein oligomer (“α-Syn”) challenge alone, in combination with various concentrations of Compound 1 (“α-Syn+Compound 1”) or in combination with 50 ng/ml positive control (“α-Syn+Pos. Control”) as compared to control (“Control”) as measured in tyrosine-hydroxylase positive (TH+) neuron count (FIG. 2A) or TH+ neurite length (FIG. 2B). Bars show mean±SEM. One way ANOVA with post hoc Dunnett's t-test: **** (p<0.0001), *** (p<0.001), ** (p<0.01), * (p<0.05), ns (not significant).

FIG. 3A-3B show effects in rat primary midbrain neuron co-cultures of alpha-synuclein oligomer (“α-Syn”) challenge alone, in combination with various concentrations of Compound 1 (“α-Syn+Compound 1”) or in combination with 50 ng/ml positive control (“α-Syn+Pos. Control”) as compared to control (“Control”) as measured in phosphorylated S129-positive TH+ neuron count (FIG. 3A) or Miro1 levels (FIG. 3B). Bars show mean±SEM. One way ANOVA with post hoc Dunnett's t-test: **** (p<0.0001), *** (p<0.001), ** (p<0.01), * (p<0.05), ns (not significant).

FIG. 4A-4C show effects in rat primary midbrain neuron co-cultures of alpha-synuclein oligomer (“α-Syn”) challenge alone, in combination with Compound 5 (“α-Syn+Compound 5”) or in combination with positive control (“α-Syn+Pos. Control”) as compared to control (“Control”) as measured in tyrosine-hydroxylase positive (TH+) neuron count (FIG. 4A), Miro1 levels (FIG. 4B), or phosphorylated alpha-synuclein (FIG. 4C). Bars show mean±SEM. One way ANOVA with post hoc Dunnett's t-test: **** (p<0.0001), *** (p<0.001), ** (p<0.01), * (p<0.05), ns (not significant).

FIG. 5A-5B show effects in rat primary midbrain neuron co-cultures of alpha-synuclein oligomer (“α-Syn”) challenge alone, in combination with various concentrations of Compound 5 (“α-Syn+Compound 5”) or in combination with 50 ng/ml positive control (“α-Syn+Pos. Control”) as compared to control (“Control”) as measured in tyrosine-hydroxylase positive (TH+) neuron count (FIG. 5A) or TH+ neurite length (FIG. 5B). Bars show mean±SEM. One way ANOVA with post hoc Dunnett's t-test: **** (p<0.0001), *** (p<0.001), ** (p<0.01), * (p<0.05), ns (not significant).

FIG. 6A-6B show effects in rat primary midbrain neuron co-cultures of alpha-synuclein oligomer (“α-Syn”) challenge alone, in combination with various concentrations of Compound 5 (“α-Syn+Compound 5”) or in combination with 50 ng/ml positive control (“α-Syn+Pos. Control”) as compared to control (“Control”) as measured in phosphorylated S129-positive TH+ neuron count (FIG. 6A) or Miro1 levels (FIG. 6B). Bars show mean±SEM. One way ANOVA with post hoc Dunnett's t-test: **** (p<0.0001), *** (p<0.001), ** (p<0.01), * (p<0.05), ns (not significant).

FIG. 7A-7B show effects in rat primary dopaminergic neuron co-cultures of alpha-synuclein oligomer (“α-Syn”) challenge alone, after treatment with various concentrations of Compound 1 (“α-Syn+Compound 1”) or after treatment with 50 ng/ml positive control (“α-Syn+Pos. Control”) as measured in phosphorylated S129-positive TH+ neuron count. FIG. 7A shows effects on neuron count after α-Syn challenge for 2 days compared to control. FIG. 7B shows effects on neuron count after α-Syn challenge for 2 days followed by treatment with Compound 1 for 2 days. Bars show mean±SEM. One way ANOVA with post hoc Dunnett's t-test: **** (p<0.0001), *** (p<0.001), ** (p<0.01), * (p<0.05), ns (not significant).

FIG. 8A-8B show effects in rat primary dopaminergic neuron co-cultures of alpha-synuclein oligomer (“α-Syn”) challenge alone, after treatment with various concentrations of Compound 1 (“α-Syn+Compound 1”) or after treatment with 50 ng/ml positive control (“α-Syn+Pos. Control”) as measured by TH+ neurite length. FIG. 8A shows effects on neurite length after α-Syn challenge for 2 days compared to control. FIG. 8B shows effects on neurite length after α-Syn challenge for 2 days followed by treatment with Compound 1 for 2 days. Bars show mean±SEM. One way ANOVA with post hoc Dunnett's t-test: **** (p<0.0001), *** (p<0.001), ** (p<0.01), * (p<0.05), ns (not significant).

FIG. 9 shows effects in rat primary dopaminergic neuron co-cultures of alpha-synuclein oligomer (“α-Syn”) challenge alone, after treatment with various concentrations of Compound 1 (“α-Syn+Compound 1”) or after treatment with 50 ng/ml positive control (“α-Syn+Pos. Control”) as measured by Miro1 protein levels in TH+ neurons after 4 days. Bars show mean±SEM. One way ANOVA with post hoc Dunnett's t-test: * (p<0.0001), * (p<0.001), ** (p<0.01), * (p<0.05), ns (not significant).

FIG. 10A-10B show effects in rat primary dopaminergic neuron co-cultures of alpha-synuclein oligomer (“α-Syn”) challenge alone, after treatment with various concentrations of Compound 9 (“α-Syn+Compound 9”) or after treatment with 50 ng/ml positive control (“α-Syn+Pos. Control”) as measured in phosphorylated S129-positive TH+ neuron count. FIG. 10A shows effects on neuron count after α-Syn challenge for 2 days compared to control. FIG. 10B shows effects on neuron count after α-Syn challenge for 2 days followed by treatment with Compound 9 for 2 days. Bars show mean±SEM. One way ANOVA with post hoc Dunnett's t-test: **** (p<0.0001), *** (p<0.001), ** (p<0.01), * (p<0.05), ns (not significant).

FIG. 11A-11B show effects in rat primary dopaminergic neuron co-cultures of alpha-synuclein oligomer (“α-Syn”) challenge alone, after treatment with various concentrations of Compound 9 (“α-Syn+Compound 9”) or after treatment with 50 ng/ml positive control (“α-Syn+Pos. Control”) as measured by TH+ neurite length. FIG. 8A shows effects on neurite length after α-Syn challenge for 2 days compared to control. FIG. 8B shows effects on neurite length after α-Syn challenge for 2 days followed by treatment with Compound 9 for 2 days. Bars show mean±SEM. One way ANOVA with post hoc Dunnett's t-test: **** (p<0.0001), *** (p<0.001), ** (p<0.01), * (p<0.05), ns (not significant).

### DETAILED DESCRIPTION OF THE INVENTION

### I. General

The present disclosure describes a method of reducing Miro1 and/or phosphorylated alpha-synuclein levels in a cell during or after cellular stress by contacting the cell with T-type calcium channel antagonist of Formula I or a ferroptosis inhibitor of Formula II.

A T-type voltage-dependent calcium channel antagonist of Formula I, such as Compound 1, or a ferroptosis inhibitor of Formula II, such as Compound 5, may be useful in reducing a Miro1 and/or phosphorylated alpha-synuclein level in diseases or conditions that would benefit from the reduction of a Miro1 and/or phosphorylated alpha-synuclein level in a cell, for example, a neuronal cell. Such diseases or conditions include neurodegenerative diseases, such as Parkinson's disease. See, Examples 7-11. In illustrative embodiments, Compound 1 of Example 1 and Compound 5 of Example 5 have each been shown to reduce Miro1 and phosphorylated Ser129 alpha-synuclein in a prophylactic rat dopaminergic neuronal study. See, Examples 7-8. Additionally, Compound 1 and Compound 9 of Example 6 each demonstrated restoration of a pre-existing loss of dopamine neurite integrity in rat midbrain dopamine neuron co-cultures previously challenged with toxic alpha-synuclein oligomers. See, Examples 10-11. The results obtained on compounds from different chemical classes suggest that the methods and uses described herein may be useful not only for neuroprotection, e.g., prevention of further loss of neuronal structure and/or function, but also neuronal recovery, e.g., restoration of already lost neuronal structure and/or function, which has previously been difficult to show.

### II. Definitions

Unless defined otherwise, all technical and scientific terms used herein have the same meaning as commonly understood by one of ordinary skill in the art.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Alkyl” is a linear or branched saturated monovalent or divalent hydrocarbon. For example, an alkyl group can have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆ alkyl) or 1 to 4 carbon atoms (i.e., (C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers an alkoxy group linked to an alkyl group which is linked to the remainder of the compound such that the alkyl group is divalent. Alkoxyalkyl can have any suitable number of carbon, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). The number of carbons refers to the total number of carbons in the alkoxy and the alkyl group. For example, C₆ alkoxyalkyl refers to ethoxy (C₂ alkoxy) linked to a butyl (C₄ alkyl), and n-propoxy (C₃ alkoxy) linked to a isopropyl (C₃ alkyl). Alkoxy and alkyl are as defined above where the alkyl is divalent, and can include, but is not limited to, methoxymethyl (CH₃OCH₂—), methoxyethyl (CH₃OCH₂CH₂—) and others.

“Halo” or “halogen” as used herein refers to fluoro (—F), chloro (—Cl), bromo (—Br) and iodo (—I).

“Haloalkyl” as used herein refers to an alkyl as defined herein, wherein one or more hydrogen atoms of the alkyl are independently replaced by a halo substituent, which may be the same or different. For example, C₁₋₄ haloalkyl is a C₁₋₄ alkyl wherein one or more of the hydrogen atoms of the C₁₋₄ alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl.

“Cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g. tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

“Heterocyclyl” or “heterocycle” or “heterocycloalkyl” as used herein refers to a single saturated or partially unsaturated non-aromatic ring or a non-aromatic multiple ring system that has at least one heteroatom in the ring (i.e., at least one annular heteroatom selected from oxygen, nitrogen, and sulfur). Unless otherwise specified, a heterocyclyl group has from 3 to about 20 annular atoms, for example from 3 to 12 annular atoms, for example from 3 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring (e.g. bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like. The heterocycle can be unsubstituted or substituted.

A “nitrogen-containing heterocyclic ring” as used herein is a monocyclic or bicyclic heterocycle that has at least one nitrogen within the ring. The nitrogen-containing heterocyclic ring can be bridged, spiro, or fused. Exemplary monocyclic nitrogen-containing heterocycles include piperidine, pyrrolidine, and piperazine. The nitrogen-containing heterocyclic ring can be unsubstituted or substituted.

A “bicyclic nitrogen-containing heterocyclic ring” as used herein is a bicyclic heterocycle that has at least one nitrogen within the ring. The bicyclic nitrogen-containing heterocyclic ring can be bridged, spiro, or fused. Exemplary bicyclic nitrogen-containing heterocycles include 3,8-diazabicyclo[3.2.1]octane, 2,5-dimethyl-2,5-diazabicyclo[2.2.2]octane, 3,9-diazabicyclo[3.3.1]nonane, 2,6-diazaspiro[3.3]heptane, and 2,5-dimethyloctahydro-1H-pyrrolo[3,4-c]pyridine. The bicyclic nitrogen-containing heterocyclic ring can be unsubstituted or substituted.

“Heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5 to 10 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, phenothiazinyl, and triazolyl. The heteroaryl can be substituted or unsubstituted.

“Phenothiazine” as used herein refers to 10H-phenothiazine, having the structure:

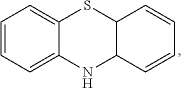 [SMILES: C1=CC2Nc3ccccc3SC2C=C1]

and tautomers thereof.

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

A “compound of the disclosure” or “compound of the present disclosure” includes compounds disclosed herein, for example a compound of the present disclosure includes compounds of Formula I and II, including the compounds of the Examples.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

“Pharmaceutically effective amount” refers to an amount of the compound of the present disclosure in a formulation or combination thereof, that provides the desired therapeutic or pharmaceutical result.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Mitochondrial Rho GTPase 1 (“MIRO1” or “Miro1”) is an enzyme that in humans is encoded by the RHOT1 gene on chromosome 17. As a Miro protein isoform, the protein facilitates mitochondrial transport by attaching the mitochondria to the motor/adaptor complex.

“Miro1 reducer” or “Miro1-reducing agent”, refers to any agent that decreases the level of a Miro1 nucleic acid, e.g., a Miro1 RNA or a Miro1 DNA, and/or a Miro1 protein in cells. In an exemplary embodiment, a Miro1-reducing agent may decrease at least one biological activity of a Miro1 protein in a cell with depolarized mitochondria. Exemplary biological activities of Miro1 include promoting mitochondrial transport, mitophagy, microtubule binding, mitochondrial fission and fusion among others. A Miro1-reducing agent can be, for example, a small molecule, a peptide, an aptamer, a protein or a functional fragment of a protein. A functional fragment of a protein, as used here, refers to all or part of the molecular elements of a protein which affect a specified function such as protein binding, signal transduction etc. In some embodiments, a Miro1 reducer and/or Miro1-reducing agent is a compound of the present disclosure, e.g., a compound of Formula (I) or Formula (II), or pharmaceutically acceptable salt thereof.

Alpha-synuclein (“α-synuclein” or “α-syn”) is a soluble unfolded protein that accumulates in Lewy bodies and Lewy neurites in Parkinson disease and other synucleinopathies. Mutations in the gene encoding α-synuclein can be linked to familial Parkinson disease. “Phosphorylated alpha-synuclein” can refer to forms of α-synuclein protein that has been phosphorylated at one or more amino acids.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of the compound that, when administered to a subject for treating a disease, is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Subject” is any mammal, such as a mouse, a rat, a dog, a cat, including veterinary animals, such as a goat, a pig, a horse, a cow, or a donkey, and primates, such as non-human primates, e.g., a cynomolgous monkey, rhesus monkey, or chimpanzee, as well as humans. In some embodiments, the subject is a human. In some embodiments, the subject is a patient.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

“Disease” or “condition” refer to a state of being or health status of a subject capable of being treated with a compound, pharmaceutical composition, or method provided herein.

### III. Compounds

The compounds of the present disclosure, or pharmaceutically acceptable salts thereof, can be used in any method to reduce Miro1 and/or phosphorylated alpha-synuclein levels described herein.

In some embodiments, the compound of the present disclosure is a compound having Formula I:

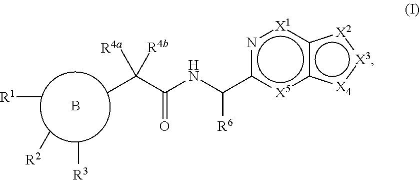

or a pharmaceutically acceptable salt thereof,
wherein
ring B is C₆-C₁₀ aryl or 5- to 10-membered heteroaryl;
R¹, R², and R³ are each independently H, halogen, CN, OR¹¹, NR^{12a}R^{12b}, C₁-C₆ alkyl, C₂-C₆ alkoxyalkyl, C₁-C₆ haloalkyl, C₂-C₆ alkenyl, C₂-C₆ alkynyl, phenyl, 5- to 6-membered heteroaryl, C₃-C₇ cycloalkyl, —(CH₂)ₘ—(C₃-C₇ cycloalkyl), 4- to 7-membered heterocyclyl, or —(CH₂)ₘ-(4- to 7-membered heterocyclyl), wherein the phenyl, heteroaryl, cycloalkyl or heterocyclyl is optionally substituted by 1, 2, or 3 halogen, CN, OR¹¹, NR^{12a}R^{12b}, C₁-C₆ alkyl, C₁-C₆ alkoxy, C₁-C₆ alkylamino, or C₁-C₆ haloalkyl;
R^{4a} and R^{4b} are each independently H or C₁-C₆ alkyl, or R^{4a} and R^{4b} and the carbon atom to which they are attached form a C₃-C₅ cycloalkyl;
R⁶ is H, C₁-C₆ alkyl, or C₁-C₆ haloalkyl;
X¹ and X⁵ are each independently N or CR⁸, provided that at least one of X¹ and X⁵ is CR⁸;
X², X³, and X⁴ are each independently N, NR⁹, O, S, or CR⁹, provided that at least one of X², X³, and X⁴ is N, NR⁹, O, or S;
R⁸ is H, halogen, CN, OR¹, C₁-C₆ alkyl, C₂-C₆ alkoxyalkyl, or C₁-C₆ haloalkyl;
R⁹ is H, C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen, CN, OH, NH₂, C₁-C₆ alkyl, C₁-C₆ alkoxy, C₁-C₆alkylamino, or C₁-C₆ haloalkyl;
each R¹¹, R^{12a}, and R^{12b} is independently H, C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen or CN; and
subscript m is 1, 2, or 3.

In some embodiments, X¹ is CR⁸.

In some embodiments, R⁸ is H.

In some embodiments, X², X³, and X⁴ are each independently N, NR⁹, or CR⁹, provided that at least one of X², X³, and X⁴ is N or NR⁹.

In some embodiments, X¹ is N or CR⁸, X² and X³ are each independently N or NR⁹, X⁴ is N, NR⁹, or CR⁹, and X⁵ is CR⁸. For instance, X¹ can be CR⁸, X² and X³ can be each independently N or NR⁹, X⁴ can be N, NR⁹, or CR⁹, and X⁵ can be CR⁸. In some embodiments, X¹ is N or CR⁸; X² is N or NR⁹; X³ is N, NR⁹, or CR⁹; X⁴ is N or CR⁹; and X⁵ is CR⁹. In some embodiments, X¹ is CR⁸; X² is N or NR⁹; X³ is N, NR⁹, or CR⁹; X⁴ is N or CR⁹; and X⁵ is CR⁹.

In some embodiments of the compound of Formula (I), or a pharmaceutically acceptable salt thereof, the compound does not have the structure:

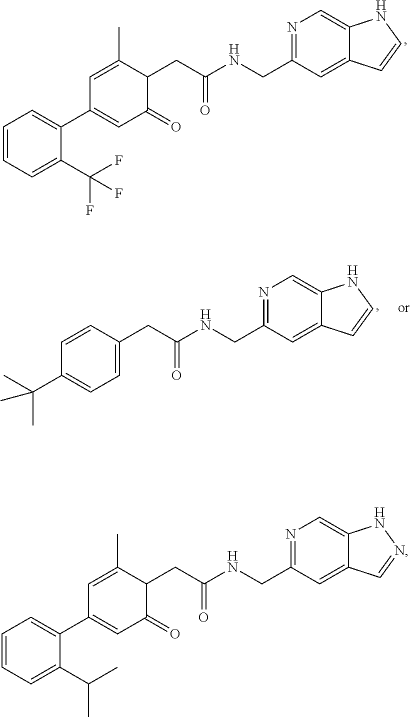 [SMILES: CC1=CC(c2ccccc2C(F)(F)F)=CC(=O)C1CC(=O)NCc1cc2cc[nH]c2cn1 ; CC(C)(C)c1ccc(CC(=O)NCc2cc3cc[nH]c3cn2)cc1 ; CC1=CC(c2ccccc2C(C)C)=CC(=O)C1CC(=O)NCc1cc2cn[nH]c2cn1]

or a pharmaceutically acceptable salt thereof.

In some embodiments, the compound has the structure of Formula (Ia):

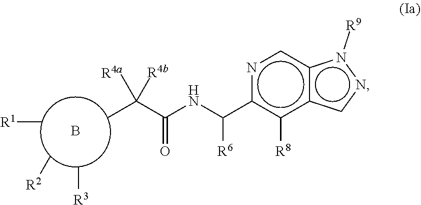

or a pharmaceutically acceptable salt thereof.

In some embodiments of the compound of Formula (I) and/or (Ia), R¹, R², and R³ are each independently H, halogen, CN, OH, NH₂, C₁-C₆ alkyl, C₁-C₆ alkoxy, C₁-C₆ alkylamino, C₂-C₆ alkoxyalkyl, C₁-C₆ haloalkyl, or C₃-C₇ cycloalkyl, wherein the cycloalkyl is optionally substituted by 1, 2, or 3 halogen, CN, OH, NH₂, C₁-C₆ alkyl, C₁-C₆ alkoxy, C₁-C₆ alkylamino, or C₁-C₆ haloalkyl.

In some embodiments of the compound of Formula (I) and/or (Ia), R³ is H, halogen, or CN. In some embodiments, R³ is H or halogen. In some embodiments, R³ is H.

In some embodiments of the compound of Formula (I) and/or (Ia), R^{4b} is H or CH₃.

In some embodiments of the compound of Formula (I) and/or (Ia), R^{4a} and R^{4b} are each independently H or CH₃.

In some embodiments of the compound of Formula (I) and/or (Ia), ring B is phenyl, 1H-benzo[d]imidazol-5-yl, 1H-indazol-5-yl, benzo[d][1,3]dioxol-5-yl, 2H-indazol-6-yl, thiophen-2-yl, pyridin-2-yl, or pyridin-3-yl; R¹ is F, Me, iPr, CF₃, CF₂CH₃, cyclopropyl, 1-fluorocyclopropyl, 1-cyanocyclopropyl, 2,2-difluorocyclopropyl, or 1-trifluoromethylcyclopropyl, 1,1-difluoromethylcyclopropyl; R² is H, F, Cl, CN, or CH₃; R³ is H or CH₃.

In some embodiments, the compound has the structure of Formula (Ib):

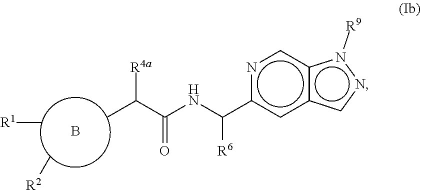

or a pharmaceutically acceptable salt thereof.

In some embodiments of the compound of Formula (I), (Ia), and/or (Ib), ring B is phenyl or 5- to 6-membered heteroaryl. In some embodiments, ring B is phenyl or pyridyl.

In some embodiments of the compound of Formula (I), (Ia), and/or (Ib), R^{4a} is H or CH₃.

In some embodiments, the compound has the structure of Formula (Ic):

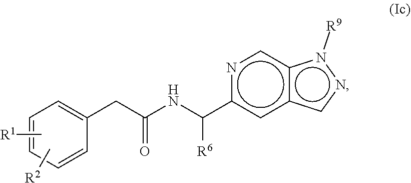

or a pharmaceutically acceptable salt thereof.

In some embodiments of the compound of Formula (I), (Ia), (Ib), and/or (Ic), R¹ is H, halogen, C₁-C₆ alkyl, C₁-C₆ alkoxy, or C₂-C₆ alkoxyalkyl. In some embodiments, R¹ is H, halogen, C₁-C₃ alkyl, or C₁-C₃ haloalkyl. In some embodiments, R¹ is H or halogen. In some embodiments, R¹ is C₁-C₃ haloalkyl. In some embodiments, R¹ is CF₃.

In some embodiments of the compound of Formula (I), (Ia), (Ib), and/or (Ic), R² is H, halogen, C₁-C₆ alkyl, C₁-C₆ alkoxy, or C₂-C₆ alkoxyalkyl. In some embodiments, R² is H, halogen, C₁-C₃ alkyl, or C₁-C₃ haloalkyl. In some embodiments, R² is H or halogen. In some embodiments, R² is H.

In some embodiments of the compound of Formula (I), (Ia), (Ib), and/or (Ic), R¹ and R² are each independently H, halogen, C₁-C₆ alkyl, C₁-C₆ alkoxy, C₂-C₆ alkoxyalkyl, C₁-C₆ haloalkyl, or C₃-C₇ cycloalkyl, wherein the cycloalkyl is optionally substituted by 1, 2, or 3 halogen, CN, OH, NH₂, C₁-C₆ alkyl, C₁-C₆ alkoxy, C₁-C₆ alkylamino, or C₁-C₆ haloalkyl. In some embodiments, R¹ and R² are each independently H, halogen, C₁-C₃ alkyl, C₁-C₃ alkoxy, C₂-C₃ alkoxyalkyl, C₁-C₃ haloalkyl, or C₃-C₅ cycloalkyl, wherein the cycloalkyl is optionally substituted by 1, 2, or 3 halogen, CN, C₁-C₃ alkyl, C₁-C₃ alkoxy, C₁-C₃ alkylamino, or C₁-C₃ haloalkyl. In some embodiments, R¹ and R² are each independently H, halogen, C₁-C₃ alkyl, or C₁-C₃ haloalkyl. In some embodiments, R¹ and R² are each independently H or halogen. In some embodiments, R¹ and R² are each independently H, C₁-C₃ alkyl, or C₁-C₃ haloalkyl. In some embodiments, R¹ and R² are each independently H or C₁-C₃ haloalkyl.

In some embodiments of the compound of Formula (I), (Ia), (Ib), and/or (Ic), R¹ is H or C₁-C₃ haloalkyl; and R² is H or halogen. In some embodiments, R¹ is C₁-C₃ haloalkyl; and R² is H. In some embodiments, R¹ is CF₃; and R² is H.

In some embodiments of the compound of Formula (I), (Ia), (Ib), and/or (Ic), R⁶ is H or C₁-C₃ alkyl. In some embodiments of the compound of Formula (I), (Ia), (Ib), and/or (Ic), R⁶ is C₁-C₃ alkyl. In some embodiments, R⁶ is CH₃.

In some embodiments of the compound of Formula (I), (Ia), (Ib), and/or (Ic), R⁹ is C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen, CN, OH, NH₂, C₁-C₆ alkyl, C₁-C₆ alkoxy, C₁-C₆ alkylamino, or C₁-C₆ haloalkyl. In some embodiments, R⁹ is C₁-C₆ alkyl, C₂-C₆ alkoxyalkyl, C₁-C₆ haloalkyl, C₃-C₇ cycloalkyl, or 4- to 7-membered heterocyclyl, wherein the cycloalkyl or heterocyclyl is substituted by 0, 1, 2, or 3 halogen, CN, OH, NH₂, C₁-C₆ alkyl, C₁-C₆ alkoxy, C₁-C₆ alkylamino, or C₁-C₆ haloalkyl. In some embodiments, R⁹ is C₁-C₃ alkyl, C₂-C₃ alkoxyalkyl, C₁-C₃ haloalkyl, C₃-C₇ cycloalkyl, or 4- to 7-membered heterocyclyl, wherein the cycloalkyl or heterocyclyl is substituted by 0, 1, 2, or 3 F, Cl, CN, OH, NH₂, C₁-C₃ alkyl, C₁-C₃ alkoxy, C₁-C₃ alkylamino, or C₁-C₃ haloalkyl. In some embodiments, R⁹ is C₁-C₃ alkyl, C₂-C₃ alkoxyalkyl, or C₁-C₃ haloalkyl. In some embodiments, R⁹ is C₁-C₃ haloalkyl. In some embodiments, R⁹ is CH₂CF₃.

In some embodiments of the compound of Formula (I), (Ia), (Ib), and/or (Ic), the compound does not have the structure:

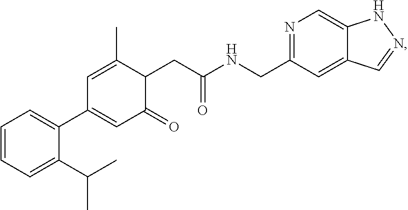 [SMILES: CC1=CC(c2ccccc2C(C)C)=CC(=O)C1CC(=O)NCc1cc2cn[nH]c2cn1]

or a pharmaceutically acceptable salt thereof.

In some embodiments of the compound of Formula (I), (Ia), (Ib), and/or (Ic), the compound has the structure:

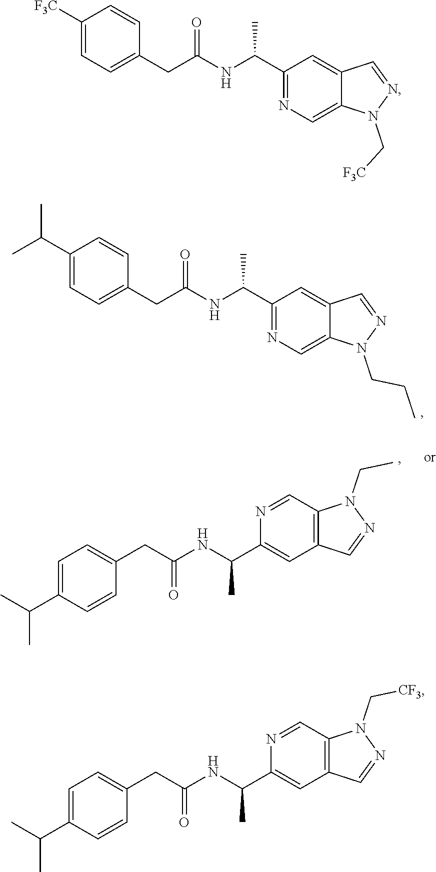

or a pharmaceutically acceptable salt thereof.

In some embodiments of the compound of Formula (I), (Ia), (Ib), and/or (Ic), the compound has the structure:

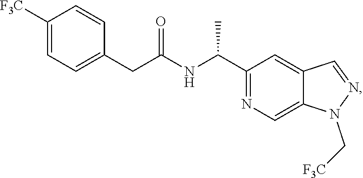 [SMILES: C[C@@H](NC(=O)Cc1ccc(C(F)(F)F)cc1)c1cc2cnn(CC(F)(F)F)c2cn1]

or a pharmaceutically acceptable salt thereof.

In some embodiments, the compound of the present disclosure is a compound having Formula II:

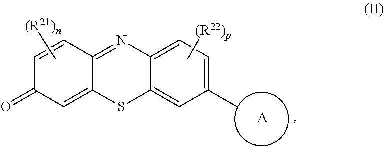

or a pharmaceutically acceptable salt thereof,
wherein
each R²¹ is independently —F, —Cl, —Br, —I, —OR^{a}, —SR^{a}, —NR^{a}R^{b}, —NO₂, —CN, C₁₋₆ alkyl, C₂₋₆ alkenyl, or C₂₋₆ alkynyl, wherein the alkyl, alkenyl, or alkynyl is substituted with 0, 1, 2, or 3 groups independently selected from —F, —Cl, —Br, —I, —OR^{a}, —SR^{a}, —NR^{a}R^{b}, oxo, —NO₂, and —CN;
each R²² is independently —F, —Cl, —Br, —I, —OH, —OR^{a}, —SR^{a}, —NR^{a}R^{b}, —CN, C₁₋₆ alkyl, C₂₋₆ alkenyl, or C₂₋₆ alkynyl, wherein the alkyl, alkenyl, or alkynyl is substituted with 0, 1, 2, or 3 groups independently selected from —F, —Cl, —Br, —I, —OR^{a}, —SR^{a}, —NR^{a}R^{b}, oxo, —NO₂, and —CN;
each R^{a} is independently H or C₁₋₆ alkyl;
each R^{b} is independently H or C₁₋₆ alkyl;
the subscript n is 0, 1, 2 or 3;
the subscript p is 0, 1, 2, or 3; and

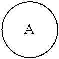

is a nitrogen-containing heterocyclic ring.

In some embodiments of the compound of Formula (II), or pharmaceutically acceptable salt thereof, each R²¹ is independently —F, —Cl, —Br, —I, —OR^{a}, —SR^{a}, —NR^{a}R^{b}, —CN, or C₁₋₆ alkyl.

In some embodiments of the compound of Formula (II), or pharmaceutically acceptable salt thereof, each R²² is independently —F, —Cl, —Br, —I, —OR^{a}, —SR^{a}, —NR^{a}R^{b}, —CN, or C₁₋₆ alkyl.

In some embodiments of the compound of Formula (II), or pharmaceutically acceptable salt thereof, each R^{a} is independently H or C₁₋₃ alkyl.

In some embodiments of the compound of Formula (II), or pharmaceutically acceptable salt thereof, each R^{b} is independently H or C₁₋₃ alkyl.

In some embodiments of the compound of Formula (II), or pharmaceutically acceptable salt thereof, the subscript m is 0 or 1.

In some embodiments of the compound of Formula (II), or pharmaceutically acceptable salt thereof, the subscript n is 0 or 1.

In some embodiments of the compound of Formula (II), or pharmaceutically acceptable salt thereof, the compound has the structure of Formula (IIa):

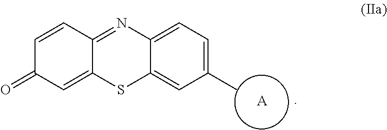

In some embodiments,

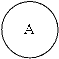

is a pyrrolidine, piperidine, or piperazine.

In some embodiments,

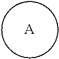

is a bicyclic nitrogen-containing heterocyclic ring.

In some embodiments of the compound of Formula (II) and/or (IIa), or pharmaceutically acceptable salt thereof,

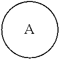

has the structure:

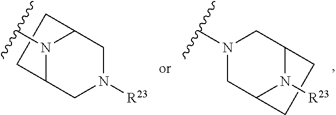

wherein
R²³ is H, C₁₋₆ alkyl, C₂₋₆ alkenyl, C₂₋₆ alkynyl, C₃₋₈ cycloalkyl, 4- to 8-membered heterocyclyl, phenyl, or 5- to 10-membered heteroaryl, wherein the alkyl, alkenyl, alkynyl, cycloalkyl, heterocyclyl, phenyl, or heteroaryl is substituted with 0, 1, 2, or 3 groups independently selected from —F, —Cl, —Br, —I, —OR^{23a}, —SR^{23a}, —NR^{23a}R^{23b}, oxo, —NO₂, and —CN;
each R^{23a} is independently H or C₁₋₆alkyl; and
each R^{23a} is independently H or C₁₋₆ alkyl.

In some embodiments of the compound of Formula (II) and/or (IIa), or pharmaceutically acceptable salt thereof, the heterocyclyl has 1, 2, or 3 atoms selected from N, O, and S.

In some embodiments of the compound of Formula (II) and/or (IIa), or pharmaceutically acceptable salt thereof, the heteroaryl has 1, 2, or 3 atoms selected from N, O, and S.

In some embodiments of the compound of Formula (II) and/or (IIa), the compound has the structure:

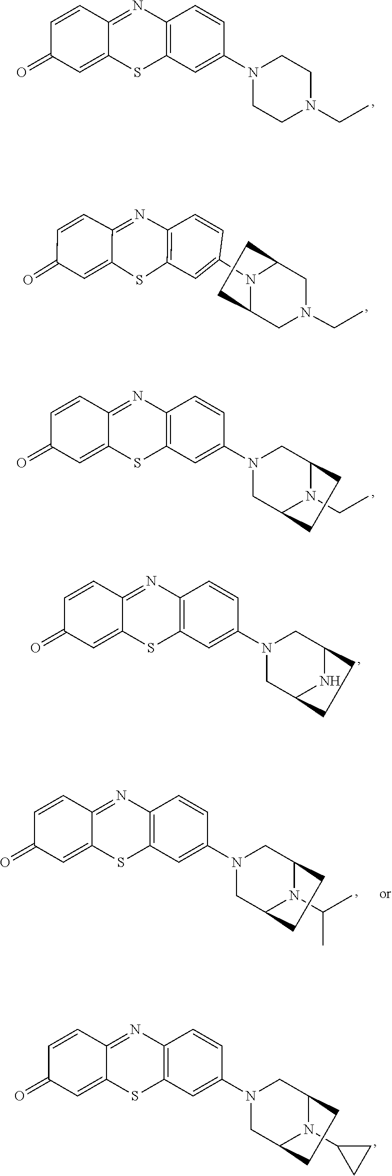 [SMILES: CCN1CCN(c2ccc3nc4ccc(=O)cc-4sc3c2)CC1 ; CCN1C[C@H]2CC[C@@H](C1)N2c1ccc2nc3ccc(=O)cc-3sc2c1 ; CCN1[C@@H]2CC[C@H]1CN(c1ccc3nc4ccc(=O)cc-4sc3c1)C2 ; O=c1ccc2nc3ccc(N4C[C@H]5CC[C@@H](C4)N5)cc3sc-2c1 ; CC(C)N1[C@@H]2CC[C@H]1CN(c1ccc3nc4ccc(=O)cc-4sc3c1)C2 ; O=c1ccc2nc3ccc(N4C[C@H]5CC[C@@H](C4)N5C4CC4)cc3sc-2c1]

or a pharmaceutically acceptable salt thereof.

In some embodiments of the compound of Formula (II) and/or (IIa), the compound has the structure:

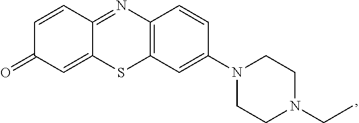 [SMILES: CCN1CCN(c2ccc3nc4ccc(=O)cc-4sc3c2)CC1]

or a pharmaceutically acceptable salt thereof.

In some embodiments of the compound of Formula (II) and/or (IIa), the compound has the structure:

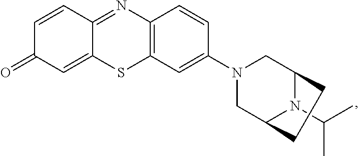 [SMILES: CC(C)N1[C@@H]2CC[C@H]1CN(c1ccc3nc4ccc(=O)cc-4sc3c1)C2]

or a pharmaceutically acceptable salt thereof.

### IV. Compositions

The disclosure provides for, inter alia, compositions of one or more compounds that are Miro1 reducers, Miro1-reducing agents, and/or reduces the level of phosphorylated alpha-synuclein as disclosed herein. The compositions of the one or more compounds can decrease the level of Miro1 and/or phosphorylated alpha-synuclein in a cell.

In some embodiments, the composition comprises a compound of the present disclosure, or a salt thereof. In some embodiments, the composition further comprises a carrier or excipient.

The compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

### A. Formulation

In some embodiments, the present disclosure provides a pharmaceutical composition, or pharmaceutical formulation, comprising a pharmaceutically effective amount of a compound of the present disclosure, or a pharmaceutically acceptable salt thereof, and a pharmaceutically acceptable carrier or excipient. The pharmaceutical composition is capable of delivering an amount of a compound of the disclosure sufficient to produce a therapeutically effective treatment as described further below. Also provided herein is a pharmaceutical formulation comprising a pharmaceutically effective amount of a compound of Formula (I) and/or (II), or a pharmaceutically acceptable salt thereof, and a pharmaceutically acceptable carrier or excipient.

For preparing pharmaceutical compositions from the compound or pharmaceutically acceptable salt of the present invention, pharmaceutically acceptable carriers can be either solid or liquid. Solid form preparations include powders, cachets, and dispersible granules. A solid carrier can be one or more substances, which may also act as diluents, binders, preservatives, disintegrating agents, or an encapsulating material.

The compounds herein are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (2006). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, comprise at least one active ingredient, as above defined, together with one or more acceptable carriers and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the administration routes described below. The formulations may conveniently be presented in unit dosage form and may be prepared by any of the methods well known in the art of pharmacy. Techniques and formulations generally are found in Remington: The Science and Practice of Pharmacy, 23^{rd} Ed. Adejare, A. et al, eds. London: Academic Press, 2020. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compound of the present invention.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Liquid form preparations include solutions, suspensions, and emulsions, for example, water or water/propylene glycol solutions. For parenteral injection, liquid preparations can be formulated in solution in aqueous polyethylene glycol solution.

Pharmaceutical formulations herein comprise a combination together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, solutions, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations suitable for topical administration to the eye also include eye drops wherein the active ingredient is dissolved or suspended in a suitable carrier, especially an aqueous solvent for the active ingredient. The active ingredient can be present in such formulations in a concentration of about 0.5 to about 20%, such as about 0.5 to about 10%, for example about 1.5% w/w.

Aqueous solutions suitable for oral use can be prepared by dissolving the compound or pharmaceutically acceptable salt of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolality.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound or pharmaceutically acceptable salt of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration into a body cavity. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV, intratumoral, or intravitreal administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, e.g., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., A1-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

### B. Administration

The compound or pharmaceutically acceptable salt and compositions of the present invention can be delivered by any suitable means, including oral, parenteral and topical methods.

A compound or composition of the present disclosure may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosage or dosing frequency of a compound or composition of the present disclosure may be adjusted over the course of the treatment, based on the judgment of the administering physician.

The compound or composition may be administered to an individual (e.g., a human) in an effective amount. In some embodiments, the compound is administered once daily.

The pharmaceutical preparation is preferably in unit dosage form. In such form the preparation is subdivided into unit doses containing appropriate quantities of the compounds and compositions of the present invention. The unit dosage form can be a packaged preparation, the package containing discrete quantities of preparation, such as packeted tablets, capsules, and powders in vials or ampoules.

Co-administration as used herein refers to administration of unit dosages of the compounds disclosed herein before, at the same time, or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

The compounds and compositions of the present invention can be co-administered with other agents. Co-administration includes administering the compound or composition of the present invention within 0.5, 1, 2, 4, 6, 8, 10, 12, 16, 20, or 24 hours of the other agent. Co-administration also includes administering simultaneously, approximately simultaneously (e.g., within about 1, 5, 10, 15, 20, or 30 minutes of each other), or sequentially in any order. Moreover, the compounds and compositions of the present invention can each be administered once a day, or two, three, or more times per day so as to provide the preferred dosage level per day.

In some embodiments, co-administration can be accomplished by co-formulation, e.g., preparing a single pharmaceutical composition including the compounds and compositions of the present invention and any other agent. Alternatively, the various components can be formulated separately.

The compounds and compositions of the present invention, and any other agents, can be present in any suitable amount, and can depend on various factors including, but not limited to, weight and age of the subject, state of the disease, etc. Suitable dosage ranges include from about 0.1 mg to about 10,000 mg, or about 1 mg to about 1000 mg, or about 10 mg to about 750 mg, or about 25 mg to about 500 mg, or about 50 mg to about 250 mg. Suitable dosages also include about 1 mg, 5, 10, 20, 30, 40, 50, 60, 70, 80, 90, 100, 200, 300, 400, 500, 600, 700, 800, 900 or 1000 mg. The composition can also contain other compatible therapeutic agents. The compounds described herein can be used in combination with one another, with other active agents known to be useful in modulating Miro1 and/or phosphorylated alpha-synuclein, or with adjunctive agents that may not be effective alone, but may contribute to the efficacy of the active agent.

### V. Methods

### A. Assays

A compound of the present disclosure capable of reducing Miro1 and/or phosphorylated alpha-synuclein level and/or activity may be validated as such by any convenient method in the art for detecting the level and/or activity of Miro1 and/or phosphorylated alpha-synuclein in the presence versus absence of the compound of the present disclosure.

In an exemplary embodiment, a Miro1-reducing agent may decrease at least one biological activity of a Miro1 protein in a cell with depolarized mitochondria. Exemplary biological activities of Miro1 include promoting mitochondrial transport, mitophagy, microtubule binding, mitochondrial fission and fusion among others. A Miro1-reducing agent can be, for example, a small molecule, a peptide, an aptamer, a protein or a functional fragment of a protein. A functional fragment of a protein, as used here, refers to all or part of the molecular elements of a protein which affect a specified function such as protein binding, signal transduction etc. In some embodiments, a Miro1 reducer and/or Miro1-reducing agent is a compound of the present disclosure, e.g., a compound of Formula (I) or Formula (II), or pharmaceutically acceptable salt thereof.

For example, the level and/or the phosphorylation state of a Miro protein (Ser156, Thr298 or Thr299 of Miro1 and Miro2, see, e.g., Wang et al. Cell 2011, 147(4): 893-906) may be detected, for example by immunoprecipitation with a mitochondrial transport protein-specific antibody followed by Western blotting with a phospho-specific or a general antibody, where an increase in phosphorylation of Miro proteins and/or a decrease of total Miro protein levels, or a decrease in phosphorylation of Khc following contact with the agent may indicate that the agent will treat Parkinson's Disease. As another example, the level and/or the ubiquitination of a Miro protein may be detected, for example by immunoprecipitation with a mitochondrial transport protein-specific antibody followed by Western blotting with a ubiquitin-specific antibody, where an increase in ubiquitination following contact with the candidate agent indicates that the agent will treat Parkinson's Disease. As another example, the ability of the target mitochondrial protein to transport mitochondria within a cell may be assessed by, for example, treating cultured cells (e.g., neurons) with the compound of the present disclosure and observing the transport of mitochondria in the cells as compared to cells not treating with the compound of the present disclosure, e.g., using live cell imaging techniques (see, e.g., Brickley and Stephenson J. Biol Chem 286(20): 18079-92 (2011); Misko et al. J Neurosci 30(19): 4232-40 (2010); Russo G J et al. J. Neurosci 29(17):5443-55 (2009)). As another example, because the formation of a complex between Miro (e.g., Miro 1 and 2), TRAK (e.g., TRAK1 and 2), and Khc is essential for mitochondrial transport in neurons (see e.g., Brickley and Stephenson J. Biol Chem 286(20): 18079-92 (2011)), the effect of the compound of the present disclosure on Miro function may be assessed by assessing the ability of Miro, TRAK and Khc to form a complex in the presence of the compound of the present disclosure. Such an assessment can be performed using any technique to determine protein-protein interaction including, but not limited to, co-immunoprecipitation and affinity purification techniques. In specific embodiments, the ability is assessed in a cell having a familial PD mutation, e.g. a PINK1 or LRRK2 mutation.

Affinity assays, which are often immunoassays, are an assay or analytic procedure that relies on the binding of the target molecule, e.g. Miro1, to receptors, antibodies or other macromolecules. A detection method is used to determine the presence and extent of the binding complexes that are formed. Many formats for such assays are known and used in the art, and are suitable for detection of Miro1 degradation following mitochondrial uncoupling or depolarization. In some embodiments, the assay format is suitable for high-throughput analysis.

Included in suitable assay formats are immunoassays that utilize antibodies specific for MiroL. Suitable antibodies for this purpose are known and commercially available as polyclonal or monoclonal compositions, e.g. from Invitrogen, including monoclonals CL1095, CL1083; from Sigma Aldrich including clone 4H4, Santa Cruz Biotechnology Anti-Rho T1 Antibody (A-8); and the like.

Assays of interest include, for example, Western blots; immunocytochemistry; immunohistochemistry; flow cytometry; immunoprecipitation; etc., and particularly include immunoassays such as enzyme-linked immunosorbent assay (ELISA), radioimmunoassay (RIA); enzyme immunoassay (EIA).

Enzyme-linked immunosorbent assays (ELISAs) are used to qualitatively and quantitatively analyze the presence or concentration of a particular soluble antigen such as Miro1, in liquid samples, such as cell lysates. These assays generally make use of the ability of multiwell plates or others to bind antibodies which trap the cognate antigen. Usually a colorimetric endpoint that can be detected via absorbance wavelength and quantitated from a known standard curve of antigen or antibody dilutions is used. The detection antibody is often labelled with an enzyme such as horseradish peroxidase or alkaline phosphatase, or a fluorescent tag, or an electrochemiluminescent label or through an intermediary label such as biotin.

Common ELISA formats include the sandwich ELISA, so named because the analyte is “sandwiched” between two different antibodies. The capture substrate in this format is a capture antibody, often a monoclonal antibody, to increase the specificity of the assay and reduce background noise. The analyte is bound to the capture antibody, then detected by binding to a detection antibody. A variation of sandwich ELISA assay, called Single-Molecule Assay (Simoa), uses beads are coated with a capture antibody; each bead is bound to either one or zero target molecule, and individual beads are detected with another antibody (detection antibody) and a labeling enzyme.

Other ELISA formats include indirect ELISA, where the capture substrate is the specific antigen that is being tested and the detection step is mediated by a primary antibody and an enzyme-conjugated secondary antibody which is reactive against the primary antibody. Thus, the primary antibody that recognizes the antigen is not labeled. In a direct ELISA the capture substrate is the specific antigen that is being tested, and the enzyme that catalyzes the color-change reaction is conjugated to the antigen detector antibody.

Immuno-PCR (I—PCR) is a technique that combines the sensitivity of the nucleic acid amplification by PCR with the specificity of the antibody-based assays resulting in an increase of the detection sensitivity.

Immunocytochemistry (ICC) is a technique that is used to visualize the localization of a specific protein or antigen in cells by use of a specific primary antibody that binds to the protein or antigen. The primary antibody allows visualization of the protein under a fluorescence microscope when it is bound by a secondary antibody that has a conjugated fluorophore. ICC permits evaluation of whether or not cells in a particular sample express the protein in question. In cases where an immunopositive signal is found, ICC also allows researchers to determine which sub-cellular compartments are expressing the antigen.

Immunohistochemistry (IHC) is an application of immunostaining. It involves the process of selectively identifying antigens (proteins) in cells of a tissue section by exploiting the principle of antibodies binding specifically to antigens in biological tissues. Visualising an antibody-antigen interaction can be accomplished in a number of ways, for example, chromogenic immunohistochemistry (CIH), wherein an antibody is conjugated to an enzyme, such as peroxidase (the combination being termed immunoperoxidase), that can catalyse a color-producing reaction, or immunofluorescence, where the antibody is tagged to a fluorophore, such as fluorescein or rhodamine.

Flow cytometry is a technology that rapidly analyzes single cells or particles as they flow past single or multiple lasers while suspended in a buffered salt-based solution. Each particle is analyzed for visible light scatter and one or multiple fluorescence parameters. Visible light scatter is measured in two different directions, the forward direction (Forward Scatter or FSC) which can indicate the relative size of the cell and at 90° (Side Scatter or SSC) which indicates the internal complexity or granularity of the cell. Light scatter is independent of fluorescence. Samples are prepared for fluorescence measurement through transfection and expression of fluorescent proteins (ex. Green Fluorescent Protein, GFP), staining with fluorescent dyes (e.g., Propidium Iodide, DNA) or staining with fluorescently conjugated antibodies (e.g., CD3 FITC).

Mass spectrometry (MS) analysis of proteins measures the mass-to-charge ratio of ions to identify and quantify molecules in simple and complex mixtures. Mass spectrometry assays can qualitatively or quantitatively measure specific analytes in complex biological matrices (such as urine, blood or tissues). Certain macromolecule ionization methods, for example, electrospray ionization (ESI) or atmospheric pressure chemical ionization (APCI), permits the study of protein structure by MS.

Calcium channel antagonist activity, including T-type calcium channel antagonist activity, can be assessed using patch clamp assays or fluorescence imaging plate reader (FLIPR) assays known in the art. See, for example, Leech, C. A. and Holz, G. G., IV. Methods Cell Biol. 1994; 40: 135-151; Bell, D. C. and Dallas, M. L. Br. J. Pharmacology 2018, 175, 2312-2321; Bezengon et al., J. Med. Chem. 2017, 60, 9769; Uebele et al., Cell Biochem Biophys 2009. 55, 81; Xiang et al, ACS Chemical Neuroscience 2011, 2, 730; and U.S. Pat. No. 10,562,857.

An exemplary method for measuring Miro1 reduction after compound administration is described in Hsieh C—H, Li L, Vanhauwaert R, Nguyen K T, Davis M D, Bu G, et al. Miro1 Marks Parkinson's Disease Subset and Miro1 Reducer Rescues Neuron Loss in Parkinson's Models. Cell Metab. 2019; 1131-1140.

Levels of any phosphorylated alpha-synuclein known in the art can be reduced by the methods of the present disclosure. In some non-limiting embodiments, phosphorylated alpha-synuclein is phosphorylated at one or more of Y125, S129, Y133, and/or Y136. In some non-limiting embodiments, phosphorylated alpha-synuclein is phosphorylated S129 alpha-synuclein.

Assays to measure phosphorylated S129 alpha-synuclein (pS129 α-syn) can be performed by any method in the art. For instance, pS129 α-syn can be measured via immunoassay (Cariulo, C. et al. Front Neurosci. 2019; 13: 889), ELISA (Majbour, et al. Mol Neurodegener. 2016; 11: 7), Western blot (Migdalska-Richards A et al. PLoS One 15:e0238075 (2020), immunocytochemistry (ICC), immunohistochemistry (IHC), Quanterix SIMOA immunoassay, mass spectroscopy, flow cytometry, or assays using Luminex technology (Wang, Y. et al. Sci Transl Med. 2012 Feb. 15; 4(121): 121ra20).

B. Methods of Reducing Miro1 and/or Phosphorylated Alpha-Synuclein

In some embodiments, a method of the present disclosure is a method of reducing Miro1 and/or phosphorylated alpha-synuclein level in a cell, comprising contacting the cell with an effective amount of any of the compounds disclosed herein, for example, a compound having Formula I or Formula II, or a pharmaceutically acceptable salt thereof. For instance, the method of reducing Miro1 and/or phosphorylated alpha-synuclein level in a cell can be performed by administering any one of the compounds of Formula I or II, or a pharmaceutically acceptable salt thereof, including the compounds of the Examples, as compared to a control cell that has not been administered a compound of the present disclosure.

In some embodiments, a reduction of a Miro1 level in a method as described herein is a reduction in the amount of a Miro1 nucleic acid, e.g., a Miro1 RNA or a Miro1 DNA, and/or a reduction in the activity of a Miro1 protein. The Miro1 nucleic acid can be any type of nucleic acid known in the art. For example, a Miro1 RNA can be a messenger RNA (mRNA), a transfer RNA (tRNA), or a ribosomal RNA (rRNA). In some embodiments, the Miro1 RNA is a messenger RNA (mRNA). In some embodiments, the reduction of a Miro1 level is a reduction in the amount of a Miro1 nucleic acid as determined by any assay method, including assays known in the art and the assays described in the present disclosure, that results in a reduction in the Miro1 activity.

In some embodiments, a reduction of a Miro1 level in a method as described herein is a reduction in the amount of a Miro1 protein and/or a reduction in the activity of a Miro1 protein. In some embodiments, the reduction of a Miro1 level is a reduction in the amount of a Miro1 protein as determined by any assay method, including assays known in the art and the assays described in the present disclosure, that results in a reduction in the Miro1 activity.

A reduction of a Miro1 and/or phosphorylated alpha-synuclein level in a method as described herein is a reduction in the amount of a Miro1 and/or phosphorylated alpha-synuclein protein and/or a reduction in the activity of a Miro1 and/or phosphorylated alpha-synuclein protein. In some embodiments, the reduction of a Miro1 and/or phosphorylated alpha-synuclein level is a reduction in the amount of a Miro1 and/or phosphorylated alpha-synuclein protein as determined by any assay method compared with controls, including assays known in the art and the assays described in the present disclosure, that results in a reduction in the Miro1 and/or phosphorylated alpha-synuclein activity. For instance, Examples 7 through 9 of the present disclosure demonstrated the reduction of Miro1 and phosphorylated S129 alpha-synuclein levels in rat neuronal cells incubated with Compound 1 or Compound 5 as compared with control cells after stress by alpha-synuclein oligomers.

A compound of the disclosure can be used in a method of reducing, or downregulating, Miro1 level in any cell. Any suitable cell can be used in a method of reducing, or downregulating, Miro1 level described herein. For example, the cell can be a primary cell or a cultured cell. Cultured cells may be derived from a human (e.g., a patient under the care of a physician) or control samples; and may be modified to generate genetically-modified cells, in vitro differentiated cells, cells exposed to a candidate therapeutic agent; and the like. In some embodiments, the cell is a neuronal cell, a muscle cell, a renal cell, a liver cell, or a skin cell. In some embodiments, the cell is a neuronal cell. In some embodiments, the cell is a muscle cell. For example, the muscle cell can be a cardiac cell, that is, a cardiomyocyte. In some embodiments, the cell is a renal cell. In some embodiments, the cell is a liver cell. In some embodiments, the cell is a skin cell, e.g., a skin fibroblast. The method can be performed in a cell in vitro, ex vivo, or in vivo. In some embodiments, the reducing Miro1 level is in vitro or ex vivo. In some embodiments, the reducing Miro1 level is in vivo.

Any suitable biological sample containing cells can be used in the methods described herein. The methods can be performed with a biological sample obtained from a subject, including without limitation biological samples such as fibroblasts, such as skin fibroblasts, peripheral blood lymphocytes, iPSCs, and the like.

A Miro1 level measured in a method described herein can be compared to a control Miro1 level by any method known in the art. See, for example, the ELISA assay described in Hsieh C—H, et al. Cell Metab. 2019; 1131-1140.

A control Miro1 and/or control phosphorylated alpha-synuclein level can be obtained from a control cell. The control cell can be a cell that has been administered only cell media (that is, without a compound of the present disclosure or pharmaceutically acceptable salt thereof). Alternatively, a control cell can be a cell from or derived from a healthy subject that does not have or is not suspected of suffering from a disease or condition described herein, e.g., a neurodegenerative disorder such as Parkinson's disease, that is administered the compound of the present disclosure or pharmaceutically acceptable salt thereof. A control cell can be obtained from or derived from a cell obtained from a healthy human volunteer or from a cell bank. For example, a control cell can be obtained from or derived from a cell obtained from American Type Culture Collection (ATCC), e.g., neural progenitor cells or neural tissue-derived cell lines such as astrocytes.

In some embodiments, control values are measured from corresponding control samples from control, e.g., non-diseased, subjects. For example, in some embodiments, a Miro1 level of a skin fibroblast from a subject, e.g., a patient, is compared to a control Miro1 level of a control skin fibroblast from a control subject.

Accordingly, in some embodiments, the reducing Miro1 and/or the phosphorylated alpha-synuclein level is compared to a control Miro1 and/or control phosphorylated alpha-synuclein level in a control cell. In some embodiments, the Miro1 and/or the phosphorylated alpha-synuclein level in a cell after administering a compound of the present disclosure, or pharmaceutically acceptable salt thereof, is from about 10% to about 95%, e.g., from about 20% to about 90%, from about 30% to about 90%, from about 20% to about 80%, from about 30% to about 80%, from about 40% to about 90%, from about 20% to about 70%, from about 20% to about 60%, or from about 30% to about 60% lower as compared to a control Miro1 and/or the control phosphorylated alpha-synuclein level in a control cell. In some embodiments, the Miro1 and/or the phosphorylated alpha-synuclein level in a cell after administering a compound of the present disclosure, or pharmaceutically acceptable salt thereof, is about 10%, 20%, 30%, 40%, 50%, 60%, 70%, 80%, 90%, or about 95% lower as compared to a control Miro1 and/or the control phosphorylated alpha-synuclein level in a control cell.

In some embodiments, the reducing Miro1 and/or the phosphorylated alpha-synuclein level is compared to a control Miro1 and/or control phosphorylated alpha-synuclein level in a control cell. In some embodiments, the Miro1 and/or the phosphorylated alpha-synuclein level in a cell after administering a compound of the present disclosure, or pharmaceutically acceptable salt thereof, is from about 10% to about 95%, e.g., from about 20% to about 90%, from about 30% to about 90%, from about 20% to about 80%, from about 30% to about 80%, from about 40% to about 90%, from about 20% to about 70%, from about 20% to about 60%, or from about 30% to about 60% higher as compared to a control Miro1 and/or the control phosphorylated alpha-synuclein level in a control cell. In some embodiments, the Miro1 and/or the phosphorylated alpha-synuclein level in a cell after administering a compound of the present disclosure, or pharmaceutically acceptable salt thereof, is about 10%, 20%, 30%, 40%, 50%, 60%, 70%, 80%, 90%, or about 95% higher as compared to a control Miro1 and/or the control phosphorylated alpha-synuclein level in a control cell.

In some embodiments, the reducing Miro1 level is compared to a control Miro1 level in a control cell. In some embodiments, the Miro1 level in a cell after administering a compound of the present disclosure, or pharmaceutically acceptable salt thereof, is from about 10% to about 95%, e.g., from about 20% to about 90%, from about 30% to about 90%, from about 20% to about 80%, from about 30% to about 80%, from about 40% to about 90%, from about 20% to about 70%, from about 20% to about 60%, or from about 30% to about 60% lower as compared to a control Miro1 level in a control cell. In some embodiments, the Miro1 level in a cell after administering a compound of the present disclosure, or pharmaceutically acceptable salt thereof, is about 10%, 20%, 30%, 40%, 50%, 60%, 70%, 80%, 90%, or about 95% lower as compared to a control Miro1 level in a control cell.

In some embodiments, the reducing Miro1 level is compared to a control Miro1 level in a control cell. In some embodiments, the Miro1 level in a cell after administering a compound of the present disclosure, or pharmaceutically acceptable salt thereof, is from about 10% to about 95%, e.g., from about 20% to about 90%, from about 30% to about 90%, from about 20% to about 80%, from about 30% to about 80%, from about 40% to about 90%, from about 20% to about 70%, from about 20% to about 60%, or from about 30% to about 60% higher as compared to a control Miro1 level in a control cell. In some embodiments, the Miro1 level in a cell after administering a compound of the present disclosure, or pharmaceutically acceptable salt thereof, is about 10%, 20%, 30%, 40%, 50%, 60%, 70%, 80%, 90%, or about 95% higher as compared to a control Miro1 level in a control cell.

In some embodiments, the phosphorylated alpha-synuclein level is compared to a control phosphorylated alpha-synuclein level in a control cell. In some embodiments, the phosphorylated alpha-synuclein level in a cell after administering a compound of the present disclosure, or pharmaceutically acceptable salt thereof, is from about 10% to about 95%, e.g., from about 20% to about 90%, from about 30% to about 90%, from about 20% to about 80%, from about 30% to about 80%, from about 40% to about 90%, from about 20% to about 70%, from about 20% to about 60%, or from about 30% to about 60% lower as compared to a control phosphorylated alpha-synuclein level in a control cell. In some embodiments, the phosphorylated alpha-synuclein level in a cell after administering a compound of the present disclosure, or pharmaceutically acceptable salt thereof, is about 10%, 20%, 30%, 40%, 50%, 60%, 70%, 80%, 90%, or about 95% lower as compared to a control phosphorylated alpha-synuclein level in a control cell.

In some embodiments, the phosphorylated alpha-synuclein level is compared to a control phosphorylated alpha-synuclein level in a control cell. In some embodiments, the phosphorylated alpha-synuclein level in a cell after administering a compound of the present disclosure, or pharmaceutically acceptable salt thereof, is from about 10% to about 95%, e.g., from about 20% to about 90%, from about 30% to about 90%, from about 20% to about 80%, from about 30% to about 80%, from about 40% to about 90%, from about 20% to about 70%, from about 20% to about 60%, or from about 30% to about 60% higher as compared to a control phosphorylated alpha-synuclein level in a control cell. In some embodiments, the phosphorylated alpha-synuclein level in a cell after administering a compound of the present disclosure, or pharmaceutically acceptable salt thereof, is about 10%, 20%, 30%, 40%, 50%, 60%, 70%, 80%, 90%, or about 95% higher as compared to a control phosphorylated alpha-synuclein level in a control cell.

C. Methods of Identifying a Subject at Risk of Developing a Disorder Associated with Elevated Levels of Miro1 and/or Phosphorylated alpha-Synuclein

A deficiency in the ability to degrade or clear Miro1 and/or phosphorylated alpha-synuclein from cells is believed to correlate with the development of a disorder associated with elevated levels of Miro1 and/or phosphorylated alpha-synuclein, for example, a neurodegenerative disorder, such as Parkinson's disease, before a subject displays an overt symptom of the neurodegenerative disorder, such as one or more of the symptoms described herein. Accordingly, in some embodiments, the subject is asymptomatic for a neurodegenerative disorder. Miro1 and/or phosphorylated alpha-synuclein may be used as a predictive biomarker for a neurodegenerative disorder in a subject at risk of developing such disorder, for example, as an initial step in treating the neurodegenerative disorder before symptoms appear. The subject at risk can have familial history of developing a neurodegenerative disorder, can present a genetic marker associated with increased risk of developing a neurodegenerative disorder, for example, LRRK2 G2019S mutation for Parkinson's disease, or can have no known risk of developing a neurodegenerative disorder.

A Miro1 and/or phosphorylated alpha-synuclein level in cells treated with a mitochondrial stressor is expected to be lower than a control Miro1 and/or phosphorylated alpha-synuclein level in untreated control cells due to mitophagy processes induced by the mitochondrial stressor. With biological sample cells derived from a subject, a Miro1 and/or phosphorylated alpha-synuclein level that is similar or higher in cells treated with a mitochondrial stressor compared to a control Miro1 and/or phosphorylated alpha-synuclein level in untreated control cells may indicate a neurodegenerative disorder that correlates with defective mitophagy processes.

Accordingly, in some embodiments, the method of the present disclosure is a method to determine the Miro1 and/or phosphorylated alpha-synuclein status of a subject comprising measuring the Miro1 and/or phosphorylated alpha-synuclein response to mitochondrial depolarization using mass spectrometry, immunocytochemistry (ICC), immunohistochemistry (IHC), flow cytometry, biochemical assays, western blotting or ELISA, to determine if a subject is deficient in the removal of Miro1 and/or phosphorylated alpha-synuclein following depolarization, wherein a subject deficient in the removal of Miro1 and/or phosphorylated alpha-synuclein following depolarization is selected for treatment by administration of a Miro1 reducer. In some embodiments, the method comprises detecting Miro1 and/or phosphorylated alpha-synuclein level in the subject, and comparing the Miro1 and/or phosphorylated alpha-synuclein level to a control Miro1 and/or phosphorylated alpha-synuclein level from a control subject. In some embodiments, the detecting Miro1 and/or phosphorylated alpha-synuclein level comprises detecting Miro1 and/or phosphorylated alpha-synuclein in a sample in the subject. In some embodiments, the sample comprises a skin cell. In some embodiments, the Miro1 and/or phosphorylated alpha-synuclein level in the subject is higher than the control Miro1 and/or phosphorylated alpha-synuclein level from a control subject. In some embodiments, the Miro1 and/or phosphorylated alpha-synuclein level in the subject is about 20% or more than the control Miro1 and/or phosphorylated alpha-synuclein level from a control subject. In some embodiments, the method comprises diagnosing the subject with a neurodegenerative disorder. In some embodiments, the neurodegenerative disorder is Parkinson's disease. In some embodiments, the method further comprises treating the subject with the Miro1 reducer. In some embodiments, method further comprises monitoring Miro1 and/or phosphorylated alpha-synuclein levels before and after treatment with the Miro1 reducer.

In some embodiments, the method of the present disclosure is a method for identifying a subject at risk of developing a disorder associated with elevated levels of Miro1 and/or phosphorylated alpha-synuclein, the method comprising: a) detecting whether a Miro1 and/or phosphorylated alpha-synuclein level is similar or higher in a biological sample obtained from the subject and treated with a mitochondrial stressor, as compared to a control Miro1 and/or phosphorylated alpha-synuclein level in a control biological sample obtained from the subject and is untreated; and b) identifying the subject at risk of developing a disorder associated with elevated levels of Miro1 and/or phosphorylated alpha-synuclein if the Miro1 and/or phosphorylated alpha-synuclein level is similar or higher in the biological sample compared to the control Miro1 and/or phosphorylated alpha-synuclein level in the control biological sample, wherein the biological sample and the control biological sample comprise iPSCs or cells differentiated from iPSCs. In some embodiments, the method further comprises treating the subject at risk of developing a disorder associated with elevated levels of Miro1 and/or phosphorylated alpha-synuclein by administering a therapeutically effective amount of a compound of the present disclosure described herein, or pharmaceutically acceptable salt thereof. In certain embodiments, the compound is a compound as described in Table 1, or a pharmaceutically acceptable salt thereof.

In some embodiments, provided is a method for identifying a subject at risk of developing a disorder associated with elevated levels of Miro1 and/or phosphorylated alpha-synuclein, the method comprising: a) detecting whether a Miro1 and/or phosphorylated alpha-synuclein level is similar or higher in a biological sample obtained from the subject and treated with a mitochondrial stressor, as compared to a control Miro1 and/or phosphorylated alpha-synuclein level in a control biological sample obtained from the subject and is untreated; b) identifying the subject at risk of developing the disorder if the Miro1 and/or phosphorylated alpha-synuclein level is similar or higher in the biological sample compared to the control Miro1 and/or phosphorylated alpha-synuclein level in the control biological sample. In some embodiments, the ratio of the Miro1 and/or phosphorylated alpha-synuclein level to the control Miro1 and/or phosphorylated alpha-synuclein level is from about 0.5 to about 10, such as from about 0.7 to about 4. In some embodiments, the method comprises treating the subject at risk of developing a disorder associated with elevated levels of Miro1 and/or phosphorylated alpha-synuclein by administering a therapeutically effective amount of a compound described herein, or pharmaceutically acceptable salt thereof. In some embodiments, the compound is a compound of Formula (I) and/or (II), or pharmaceutically acceptable salt thereof. In certain embodiments, the compound is a compound as described in Table 1, or a pharmaceutically acceptable salt thereof.

A disorder associated with elevated levels of Miro1 and/or phosphorylated alpha-synuclein is any disease or disorder that correlates with abnormal degradation and/or clearance of a Miro1 and/or phosphorylated alpha-synuclein, and includes any one of the neurodegenerative disorders described herein. In some embodiments, the disorder is Drug-induced Parkinsonism, Progressive supranuclear Palsy, Vascular Parkinsonism, Dementia with Lewy Bodies, diffuse Lewy body disease, Corticobasal degeneration, multisystem degeneration (Shy-drager syndrome), Parkinson's disease, Alzheimer's disease, Pick's disease, frontotemporal dementia, multiple systems atrophy, vascular dementia, or progressive supranuclear palsy (Steel-Richardson syndrome). In some embodiments, the disorder is Parkinson's disease. In some embodiments, the disorder is Alzheimer's disease. In some embodiments, the disorder is Pick's disease. In some embodiments, the disorder is frontotemporal dementia. In some embodiments, the disorder is multiple systems atrophy.

Further provided herein is a method for treating a neurodegenerative disorder in a subject in need thereof, the method comprising: a) detecting whether a Miro1 and/or phosphorylated alpha-synuclein level is similar or higher in a biological sample obtained from the subject and treated with a mitochondrial stressor, as compared to a control Miro1 and/or phosphorylated alpha-synuclein level in a control biological sample obtained from the subject and is untreated; b) identifying the subject for treatment if the Miro1 and/or phosphorylated alpha-synuclein level is similar or higher in the biological sample compared to the control Miro1 and/or phosphorylated alpha-synuclein level in the control biological sample; and c) administering a therapeutically effective amount of a compound as described herein, or pharmaceutically acceptable salt thereof, to the subject. In some embodiments, the neurodegenerative disorder is Drug-induced Parkinsonism, Progressive supranuclear Palsy, Vascular Parkinsonism, Dementia with Lewy Bodies, diffuse Lewy body disease, Corticobasal degeneration, multisystem degeneration (Shy-drager syndrome), Parkinson's disease, Alzheimer's disease, Pick's disease, frontotemporal dementia, multiple systems atrophy, vascular dementia, or progressive supranuclear palsy (Steel-Richardson syndrome). In some embodiments, the compound is a compound of Formula (I) and/or (II), or pharmaceutically acceptable salt thereof. In certain embodiments, the compound is a compound as described in Table 1, or a pharmaceutically acceptable salt thereof.

Any suitable biological sample described herein, including urine, tissue, cerebrospinal fluid (CSF), or blood, can be used in the methods. In some embodiments, the biological sample and the control biological sample comprise fibroblasts. For example, skin fibroblasts can be directly obtained from the subject. In some embodiments, the biological sample and the control biological sample comprise iPSCs or cells differentiated from iPSCs.

iPSCs can be directly obtained from a subject or be cultured from other cell types obtained from a subject according to any method known in the art. See, Shi, Y. et al. Nature Reviews Drug Discovery vol. 16, pages 115-130 (2017), and references cited therein. For example, iPSCs can be dedifferentiated from fibroblast cells that were directly obtained from a subject. Additionally, iPSCs can be redifferentiated into a variety of different cell types, including neuronal cells, glial cells, skin cells, blood cells, muscle cells, such as cardiac muscle cells, and liver cells.

Such cells differentiated from iPSCs of a subject can be used to determine a personalized therapy in a convenient manner without directly obtaining a target cell type directly from a subject. In an illustrative example, a skin fibroblast can be obtained from a subject at risk for developing Parkinson's disease. The skin fibroblast can be dedifferentiated into iPSCs, which can then be redifferentiated into motor neurons. The motor neurons differentiated from iPSCs can be tested in an assay described herein for Miro1 deficit with and without treatment of a mitochondrial stressor in order to identify whether the subject may be responsive to a Miro1 and/or phosphorylated alpha-synuclein reducing therapy, such as one containing a compound of the disclosure.

Methods for comparing a Miro1 and/or phosphorylated alpha-synuclein level to a control Miro1 and/or phosphorylated alpha-synuclein level are known in the art. When detecting whether a Miro1 and/or phosphorylated alpha-synuclein level is similar or higher in a biological sample obtained from the subject and treated with a mitochondrial stressor, as compared to a control Miro1 and/or phosphorylated alpha-synuclein level in a control biological sample obtained from the subject and is untreated, the ratio of the Miro1 and/or phosphorylated alpha-synuclein level to the control Miro1 and/or phosphorylated alpha-synuclein level can be compared. In some embodiments, the ratio of the Miro1 and/or phosphorylated alpha-synuclein level to the control Miro1 and/or phosphorylated alpha-synuclein level is from about 0.5 to about 10, such as from about 0.5 to about 5, from about 0.6 to about 6, from about 0.7 to about 4, from about 0.7 to about 3, from about 0.8 to about 3, or from about 0.9 to about 2. For example, the ratio of the Miro1 and/or phosphorylated alpha-synuclein level to the control Miro1 and/or phosphorylated alpha-synuclein level can be from about 0.5 to about 10. In another example, the ratio of the Miro1 and/or phosphorylated alpha-synuclein level to the control Miro1 and/or phosphorylated alpha-synuclein level can be from about 0.7 to about 4.

Any mitochondrial stressor known in the art may be used in the methods described herein. Suitable mitochondrial stressors include mitochondrial depolarizing agents, such as carbonyl cyanide 4-(trifluoromethoxy)phenylhydrazone (FCCP) and carbonyl cyanide 3-chlorophenylhydrazone (CCCP); mitochondrial electron transport chain inhibitors, including Complex I inhibitors, such as rotenone, piericidin A, 1-methyl-4-phenyl-1,2,3,6-tetrahydropyridine (MPTP), and paraquat, Complex III inhibitors, such as antimycin A, Complex V inhibitors, such as oligomycin A, and mitochondrial membrane potassium ionophores, such as valinomycin; metabolic modulators, including modulators of insulin signaling, such as metformin, and inhibitors of mTOR master signaling pathway required for cell growth and metabolism, such as rapamycin. In some embodiments, the mitochondrial stressor comprises antimycin A or carbonyl cyanide 3-chlorophenylhydrazone (CCCP). In some embodiments, the mitochondrial stressor comprises carbonyl cyanide 4-(trifluoromethoxy)phenylhydrazone (FCCP).

D. Methods and/or Uses of Treatment

As demonstrated in the Examples below, illustrative compounds of Formula (I) and (II) provided biological effects that suggest they may be useful for treating a neurodegenerative disorder. In illustrative embodiments, Compound 1 of Example 1 and Compound 5 of Example 5 have each been shown to reduce Miro1 and phosphorylated Ser129 alpha-synuclein in a prophylactic rat dopaminergic neuronal study. See, Examples 7-8. Compound 1 and Compound 9 demonstrated restoration of a pre-existing loss of dopamine neurite integrity in rat midbrain dopamine neuron co-cultures previously challenged with toxic alpha-synuclein oligomers. See, Examples 10-11. The results obtained on compounds from different chemical classes suggest that the methods and uses described herein may be useful for neuroprotection, e.g., prevention of further loss of neuronal structure and/or function, and also neuronal recovery, e.g., restoration of already lost neuronal structure and/or function.

In some embodiments, the method of the present disclosure is a method of treating a disease or condition characterized by an elevated Miro1 and/or phosphorylated alpha-synuclein level in a subject in need thereof, comprising administering to the subject a therapeutically effective amount of a compound of the present disclosure, e.g., a compound of Formula (I) or (II), or a pharmaceutically acceptable salt thereof. In some embodiments, the disease or condition comprises a neurodegenerative disorder. In some embodiments, the method comprises neuroprotection. In some embodiments, the method comprises neuronal recovery.

In some embodiments, the method of the present disclosure is a method of treating a disease or condition characterized by an elevated Miro1 and/or phosphorylated alpha-synuclein level in a subject in need thereof, comprising administering to the subject a therapeutically effective amount of a compound of Formula (I) or (II), or a pharmaceutically acceptable salt thereof. In some embodiments, the disease or condition characterized by an elevated Miro1 and/or phosphorylated alpha-synuclein level is a neurodegenerative disorder. In some embodiments, the method delivers a therapeutically effective amount of a compound of the disclosure, or a pharmaceutical composition thereof, sufficient to treat one or more symptoms of a condition described further below.

In some embodiments, the use of the present disclosure is a use in the manufacture of a medicament in treating a disease or condition characterized by an elevated Miro1 and/or phosphorylated alpha-synuclein level in a subject in need thereof, wherein the medicament comprises a therapeutically effective amount of a compound of Formula (I) or (II), or a pharmaceutically acceptable salt thereof. In some embodiments, the disease or condition characterized by an elevated Miro1 and/or phosphorylated alpha-synuclein level is a neurodegenerative disorder. In some embodiments, the use delivers a therapeutically effective amount of a compound of the disclosure, or a pharmaceutical composition thereof, sufficient to treat one or more symptoms of a condition described further below.

Neurodegenerative disorders within the methods of the present disclosure include, but are not limited to Parkinson's disease and other neurological disorders that share symptoms similar to those seen in Parkinson's disease related disorders. In some cases, the neurological disorders may show symptoms similar to Parkinson's disease, atypical Parkinson's disease or Parkinson's plus disease. Examples include but are not limited to Drug-induced Parkinsonism, Progressive supranuclear Palsy, Vascular Parkinsonism, Dementia with Lewy Bodies, diffuse Lewy body disease, Corticobasal degeneration, multisystem degeneration (Shy-drager syndrome), Alzheimer's disease, Pick's disease, frontotemporal dementia, multiple systems atrophy, vascular dementia, and progressive supranuclear palsy (Steel-Richardson syndrome). Accordingly, in some embodiments, the disease or condition characterized by an elevated Miro1 and/or phosphorylated alpha-synuclein level is Parkinson's disease, Progressive supranuclear Palsy, Vascular Parkinsonism, Dementia with Lewy Bodies, diffuse Lewy body disease, Corticobasal degeneration, multisystem degeneration (Shy-drager syndrome), Alzheimer's disease, Pick's disease, frontotemporal dementia, multiple systems atrophy, vascular dementia, or progressive supranuclear palsy (Steel-Richardson syndrome). Other conditions also included within the methods of the present invention include age-related dementia and other dementias and conditions with memory loss including vascular dementia, diffuse white matter disease (Binswanger's disease), dementia of endocrine or metabolic origin, dementia of head trauma and diffuse brain damage, dementia pugilistica and frontal lobe dementia. In some cases, the neurological disorder may not respond well to dopaminergic treatments and may be caused as a result of various vascular, drug-related, infectious, toxic, structural and other known secondary causes. Drug-induced Parkinsonism may be caused by agents that block post-synaptic dopamine D2 receptors with high affinity, such as anti-psychotic and anti-emetic medications and sodium valproate, anti-depressants, reserpine, tetrabenazine etc.

A variety of human subjects are suitable for treatment with a compound of the present disclosure, such as a compound of Formula (I) or (II), or pharmaceutically acceptable salt thereof. Suitable subjects include any subject who displays symptoms of Parkinson's disease such as bradykinesia, repetitive movements, tremors, limb rigidity, gait and balance problems, inability to aim the eyes due to weakness of eye muscles, weakness, sensory loss, non-motor manifestations such as REM sleep behavior disorder, neuropsychiatric symptoms including mood disturbances and cognitive changes, anxiety, apathy, changes in thinking ability, level of attention or alertness and visual hallucinations, intellectual and functional deterioration, forgetfulness, personality changes, autonomic dysfunction affecting cardiovascular, respiratory, urogenital, gastrointestinal and sudomotor function, difficulties in breathing and swallowing, inability to sweat, orthostatic hypotension, pain, constipation, and loss of olfaction, e.g., hyposmia. In some embodiments, the subjects may experience predominant speech or language disorder, predominant frontal presentation and gait freezing.

In some embodiments, the subject may not display any overt symptoms of Parkinson's disease. In some cases, the subject in need may show increased susceptibility to infections, hypothermia, weaker bones, joint stiffness, arthritis, stooped posture, slowed movements, decrease in overall energy, constipation, urinary incontinence, memory loss, slower thinking, slower reflexes, difficulty with balance, decrease in visual acuity, diminished peripheral vision, hearing loss, wrinkling skin, greying hair, weight loss, loss of muscle tissue.

In some embodiments, the subject is selected from those that have been diagnosed as having Alzheimer's disease; subjects who have suffered one or more strokes; subjects who have suffered traumatic head injury; individuals who have high serum cholesterol levels; subjects who have proteinopathies including deposits in brain tissue; subjects who have had one or more cardiac events; subjects undergoing cardiac surgery; and subjects with multiple sclerosis.

In some embodiments, the subject displays symptoms associated with neurological diseases that include motor neuron diseases such as amyotrophic lateral sclerosis, degenerative ataxias, cortical basal degeneration, ALS-Parkinson's-Dementia complex of Guam, subacute sclerosing panencephalitis, Huntington's disease, Alzheimer's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia). Hence, in some embodiments, the disease or condition characterized by an elevated Miro1 and/or phosphorylated alpha-synuclein level is selected from myotrophic lateral sclerosis, degenerative ataxias, cortical basal degeneration, ALS-Parkinson's-Dementia complex of Guam, subacute sclerosing panencephalitis, Huntington's disease, Alzheimer's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia). Also other neurodegenerative disorders resulting from cerebral ischemia or infaction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration).

Compounds of the disclosure or pharmaceutically acceptable salts thereof are also useful for methods of aiding the treatment of a disease, a disorder, and/or a health condition associated with elevated Miro1 and/or phosphorylated alpha-synuclein. As used herein, a “method of aiding” generally refers to methods of assisting in performing or practicing a method disclosed herein, for example, methods of assisting in (i) performing, (ii) practicing, and/or (iii) making a determination concerning the detection, classification, treatment regiment, or nature, of a disease or condition characterized by an elevated Miro1 and/or phosphorylated alpha-synuclein level (e.g., a neurodegenerative disorder such as Parkinson's disease).

Accordingly, in some embodiments, a method of aiding in the treatment of a disease or condition characterized by an elevated Miro1 and/or phosphorylated alpha-synuclein level in a subject in need thereof comprises administering to the subject a therapeutically effective amount of a compound of the present disclosure or pharmaceutically acceptable salt thereof, or a pharmaceutical composition of the present disclosure.

Kits that comprise a compound of the present disclosure, or pharmaceutically acceptable salt thereof, or a pharmaceutical composition containing any of the above, are also included in the present disclosure. In some embodiments, a kit further includes instructions for use. In some embodiments, a kit includes a compound of the disclosure, or a pharmaceutically acceptable salt thereof, and a label and/or instructions for use of the compounds in the treatment of the indications, such as the diseases or conditions, described herein. In some embodiments, kits comprising a compound of the present disclosure, or a pharmaceutically acceptable salt thereof, in combination with one or more (e.g., one, two, three, four, one or two, or one to three, or one to four) additional therapeutic agents are provided.

Provided herein are also articles of manufacture that include a compound of the present disclosure or a pharmaceutically acceptable salt thereof in a suitable container. The container may be a vial, jar, ampoule, preloaded syringe, and intravenous bag.

### VI. Examples

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

Many general references providing commonly known chemical synthetic schemes and conditions useful for synthesizing the disclosed compounds are available (see, e.g., Smith, March's Advanced Organic Chemistry: Reactions, Mechanisms, and Structure, 8^{th} edition, Wiley-Interscience, 2020.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. For example, disclosed compounds can be purified via silica gel chromatography. See, e.g., Introduction to Modern Liquid Chromatography, 3^{rd} ed., ed. L. R. Snyder and J. J. Kirkland, John Wiley and Sons, 2009; and Thin Layer Chromatography, E. Stahl (ed.), Springer-Verlag, New York, 1969.

Compounds were characterized using standard instrumentation methods. Identification of the compound was carried out by hydrogen nuclear magnetic resonance spectrum (¹H-NMR) and mass spectrum (MS). ¹H-NMR was measured at 400 MHz, unless otherwise specified. In some cases, exchangeable hydrogen could not be clearly observed depending on the compound and measurement conditions. The designation br. or broad, used herein, refers to a broad signal. HPLC preparative chromatography was carried out by a commercially available ODS column in a gradient mode using water/methanol (containing formic acid) as eluents, unless otherwise specified.

Abbreviations. Certain abbreviations and acronyms are used in describing the experimental details. Although most of these would be understood by one skilled in the art, listed below are many of these abbreviations and acronyms.

| Abbreviation | Meaning |
|---|---|
| ACN | acetonitrile |
| Bn | benzyl |
| Boc | tert-butoxycarbonyl |
| Bu | butyl |
| nBuLi | n-butyllithium |
| cBu | cyclobutyl |
| CDCl₃ | chloroform-d |
| cHex | cyclohexyl |
| cPr | cyclopropyl |
| DAPI | 4′,6-diamidino-2-phenylindole |
| DCM | dichloromethane |
| DIEA | diisopropylethylamine |
| DMAP | 4-dimethylaminopyridine |
| DMSO | dimethylsulfoxide |
| DMF | dimethylformamide |
| EA | ethyl acetate |
| EDCI | N-(3-dimethylaminopropyl)-N′-ethylcarbodiimide |
| | hydrochloride |
| ESI | electron spray ionization |
| Et | ethyl |
| HATU | hexafluorophosphate azabenzotriazole tetramethyl |
| | uronium |
| iBu | isobutyl |
| iPr | isopropyl |
| LC | liquid chromatography |
| LCMS or LC/MS | liquid chromatography-mass spectrometry |
| Me | methyl |
| m/z | mass to charge ratio |
| MS or ms | mass spectrum |
| NMP | N-methyl-2-pyrrolidone |
| Ph | phenyl |
| Ph₃P | triphenylphosphine |
| prep. HPLC | preparative high performance liquid chromatography |
| | (also prep-HPLC) |
| RT | room temperature |
| tBu | tert -butyl |
| TEA | triethylamine |
| TFA | trifluoroacetic acid |
| TH | tyrosine hydroxylase |
| TH+ | tyrosine hydroxylase positive |
| THF | tetrahydrofuran |
| TLC | thin layer chromatography |
| TMS | trimethylsilyl |
| TMSCl | trimethylsilyl chloride |
| δ | parts per million referenced to residual non-deuterated |
| | solvent peak |

### Example 1. Synthesis of (R)—N-(1-(1-(2,2,2-trifluoroethyl)-1H-pyrazolo[3,4-c]pyridin-5-yl)ethyl)-2-(4-(trifluoromethyl)phenyl)acetamide (Compound 1)

### Step 1. Synthesis of 1-(2,2,2-trifluoroethyl)-1H-pyrazolo[3,4-c]pyridine-5-carbaldehyde

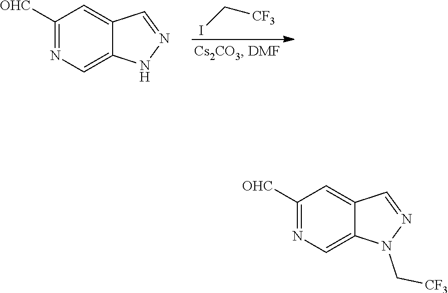

A solution of 1H-pyrazolo[3,4-c]pyridine-5-carbaldehyde (760 mg), 1,1,1-trifluoro-2-iodoethane (0.51 mL) and Cs₂CO₃ (2.5 g) in DMF (10 mL) was stirred at 100° C. for 12 hr. The mixture was diluted with water and extracted with EtOAc. The combined extracts were dried, filtered and concentrated to give a residue. The residue was purified by column chromatography on silica gel (DCM:MeOH=1:0 to 20:1) to give 1-(2,2,2-trifluoroethyl)-1H-pyrazolo[3,4-c]pyridine-5-carbaldehyde (500 mg) as a yellow solid. LC/MS ESI (m/z): 230 [M+H]⁺.

### Step 2. Synthesis of (R,E)-2-methyl-N-((1-(2,2,2-trifluoroethyl)-1H-pyrazolo[3,4-c]pyridin-5-yl)methylene)propane-2-sulfinamide

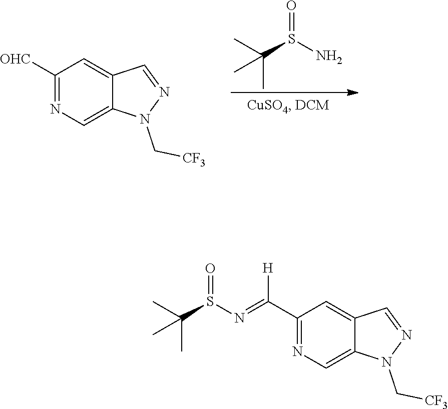

A solution of 1-(2,2,2-trifluoroethyl)-1H-pyrazolo[3,4-c]pyridine-5-carbaldehyde (500 mg), (R)-2-methylpropane-2-sulfinamide (396.7 mg) and copper sulfate pentahydrate (1.0 g) in DCM (15 mL) was stirred at 25° C. for 12 hr. The mixture was filtered and concentrated to give a residue. The residue was purified by column chromatography on silica gel (DCM:MeOH=1:0 to 20:1) to give (R,E)-2-methyl-N-((1-(2,2,2-trifluoroethyl)-1H-pyrazolo[3,4-c]pyridin-5-yl)methylene)propane-2-sulfinamide (450 mg) as a yellow solid. LC/MS ESI (m/z): 333 [M+H]⁺.

### Step 3. Synthesis of (R)-2-methyl-N—((R)-1-(1-(2,2,2-trifluoroethyl)-1H-pyrazolo[3,4-c]pyridin-5-yl)ethyl)propane-2-sulfinamide

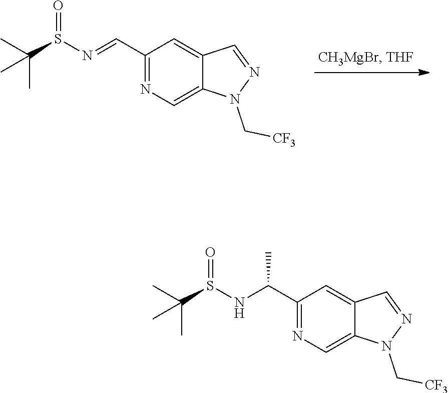 [SMILES: CC(C)(C)[S@@](=O)/N=C/c1cc2cnn(CC(F)(F)F)c2cn1 ; C[C@@H](N[S@](=O)C(C)(C)C)c1cc2cnn(CC(F)(F)F)c2cn1]

To a solution of (R,E)-2-methyl-N-((1-(2,2,2-trifluoroethyl)-1H-pyrazolo[3,4-c]pyridin-5-yl)methylene)propane-2-sulfinamide (450 mg) in THF (5 mL) was added CH₃MgBr (3.0M in ether, 1.35 mL) at −78° C. The mixture was stirred at −78° C. for 2 hr. The mixture was quenched with NH₄Cl solution and extracted with EtOAc. The combined extracts were dried, filtered and concentrated to give a residue. The residue was purified by column chromatography on silica gel (DCM:MeOH=1:0 to 20:1) to give (R)-2-methyl-N—((R)-1-(1-(2,2,2-trifluoroethyl)-1H-pyrazolo[3,4-c]pyridin-5-yl)ethyl)propane-2-sulfinamide (350 mg) as a yellow solid. LC/MS ESI (m/z): 349 [M+H]⁺.

### Step 4. Synthesis of (R)-1-(1-(2,2,2-trifluoroethyl)-1H-pyrazolo[3,4-c]pyridin-5-yl)ethan-1-amine hydrochloride

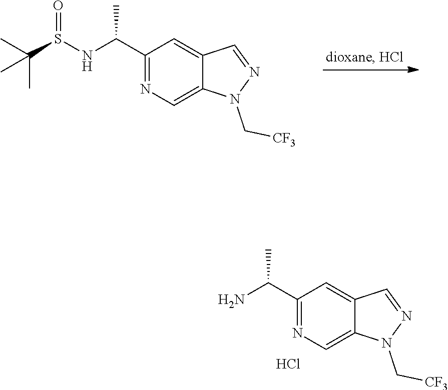 [SMILES: C[C@@H](N[S@](=O)C(C)(C)C)c1cc2cnn(CC(F)(F)F)c2cn1 ; C[C@@H](N)c1cc2cnn(CC(F)(F)F)c2cn1 ; Cl]

To a solution of (R)-2-methyl-N—((R)-1-(1-(2,2,2-trifluoroethyl)-1H-pyrazolo[3,4-c]pyridin-5-yl)ethyl)propane-2-sulfinamide (300 mg) in dioxane (2.0 mL) was added HCl/dioxane (4N in dioxane, 2.0 mL). The mixture was stirred at 25° C. for 30 min. The mixture was concentrated to give (R)-1-(1-(2,2,2-trifluoroethyl)-1H-pyrazolo[3,4-c]pyridin-5-yl)ethan-1-amine hydrochloride (300 mg, crude) as a white solid. LC/MS ESI (m/z): 245 [M+H]⁺.

### Synthesis of (R)—N-(1-(1-(2,2,2-trifluoroethyl)-1H-pyrazolo[3,4-c]pyridin-5-yl)ethyl)-2-(4-(trifluoromethyl)phenyl)acetamide (Compound 1)

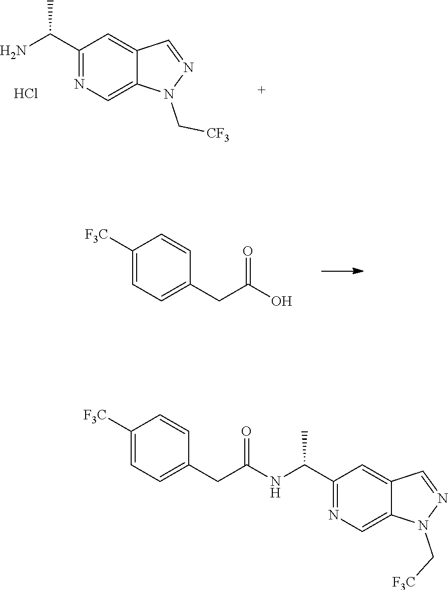 [SMILES: Cl ; C[C@@H](N)c1cc2cnn(CC(F)(F)F)c2cn1 ; O=C(O)Cc1ccc(C(F)(F)F)cc1 ; C[C@@H](NC(=O)Cc1ccc(C(F)(F)F)cc1)c1cc2cnn(CC(F)(F)F)c2cn1]

A solution of 2-(4-(trifluoromethyl)phenyl)acetic acid (96.4 mg) and HATU (269.5 mg) in 3 mL DMF was stirred at room temperature for 15 min (Solution A). DIEA (0.48 mL) was added to (R)-1-[1-(2,2,2-trifluoroethyl)-1H-pyrazolo[3,4-c]pyridin-5-yl]ethan-1-amine hydrochloride from Step 4 (150 mg) in 2 mL DMF until the pH of the solution higher than 7 by wet pH paper (Solution B). Solution B was added to Solution A, and the reaction was stirred for 1 hour, at which time LCMS showed the reaction was complete. The reaction was diluted with EA and water. The two phases were separated, the aqueous phase was extracted with DCM (10 mL×2). The combined organic layers were dried over Na₂SO₄, filtered, and concentrated in vacuo. The residue was purified by silica gel column chromatography, eluting with methanol in DCM (0-7%) to afford a crude product, which was purified by prep-HPLC (Column: YMC-Actus Triart C18 250*20 mm; Mobile phase: from 28% to 95% MeCN with H2O (0.1% formic acid); flow rate: 15 mL/min; wavelength: 220 nm/254 nm) and SFC (Column:ChiralPak IA, 250×21.2 mm I.D., 5 μm; Mobile phase: A for CO2 and B for MEOH+0.1% NH3H2O;Gradient: B 40%;Flow rate: 50 mL/min; Wavelength: 210 nm) to give N-[(1R)-1-[1-(2,2,2-trifluoroethyl)-1H-pyrazolo[3,4-c]pyridin-5-yl]ethyl]-2-[4-(trifluoromethyl)phenyl]acetamide (92.1 mg) as white solid. LC/MS ESI (m/z): 431 [M+H]⁺. ¹H-NMR (400 MHz, CDCl₃): δ 8.87 (s, 1H), 8.10 (s, 1H), 7.63-7.51 (m, 3H), 7.40 (d, J=8.0 Hz, 2H), 6.72 (d, J=6.9 Hz, 1H), 5.26-5.24 (m, 1H), 5.04 (q, J=8.3 Hz, 2H), 3.63 (s, 2H), 1.48 (d, J=6.8 Hz, 3H). ¹⁹F-NMR (377 MHz, CDCl₃): δ −62.54, −70.81.

### Example 2. (R)-2-(4-isopropylphenyl)-N-(1-(1-propyl-1H-pyrazolo[3,4-c]pyridin-5-yl)ethyl)acetamide

### Synthesis of methyl 1-propyl-1H-pyrazolo[3,4-c]pyridine-5-carbaldehyde

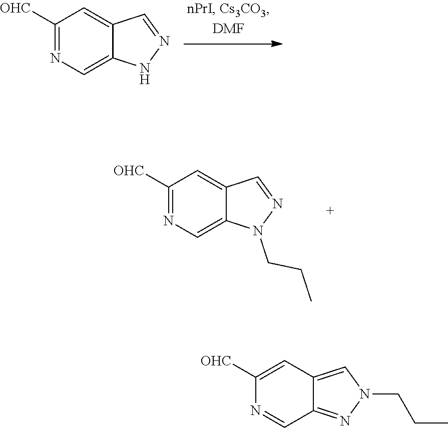

A solution of 1H-pyrazolo[3,4-c]pyridine-5-carbaldehyde (480 mg), 1-iodopropane (0.35 mL) and Cs₂CO₃ (1.6 g) in DMF (1.0 mL) was stirred at 25° C. for 12 hr. The mixture was diluted with water and extracted with EtOAc. The combined extracts were dried, filtered and concentrated to give a residue which was purified by column chromatography on silica gel (DCM:MeOH=1:0 to 20:1) to give 1-propyl-1H-pyrazolo[3,4-c]pyridine-5-carbaldehyde (185 mg) as a white solid. ¹H NMR (400 MHz, CDCl₃) δ 10.22 (s, 1H), 9.06 (s, 1H), 8.39 (d, J=1.2 Hz, 1H), 8.24 (s, 1H), 4.51 (t, J=7.2 Hz, 2H), 2.06-2.04 (m, 2H), 0.96 (t, J=7.2 Hz, 3H). and 2-propyl-2H-pyrazolo[3,4-c]pyridine-5-carbaldehyde (125 mg) as a white solid. ¹H NMR (400 MHz, CDCl₃): δ 10.18 (s, 1H), 9.32 (s, 1H), 8.32 (d, J=1.2 Hz, 1H), 8.20 (s, 1H), 4.49 (t, J=7.2 Hz, 2H), 2.12-2.08 (m, 2H), 0.98 (t, J=7.2 Hz, 3H).

### Synthesis of (R,E)-2-methyl-N-((1-propyl-1H-pyrazolo[3,4-c]pyridin-5-yl)methylene)propane-2-sulfinamide

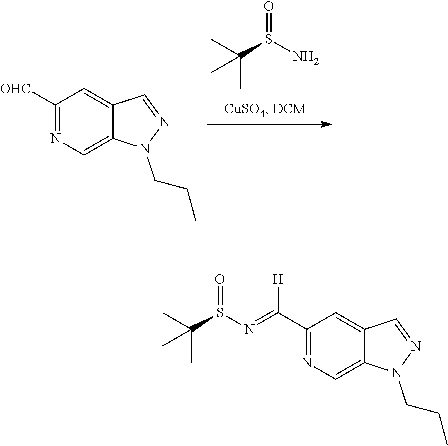

To a solution of 1-propyl-1H-pyrazolo[3,4-c]pyridine-5-carbaldehyde (185 mg) in anhydrous DCM (11 mL) were added (R)-2-methylpropane-2-sulfinamide (154.0 mg) and CuSO₄ (234.1 mg). The reaction was stirred at RT for overnight. The solid was filtered with Celite. Then, the filtrate was concentrated to provide a residue, which was purified by silica gel column chromatography to give (R,E)-2-methyl-N-((1-propyl-1H-pyrazolo[3,4-c]pyridin-5-yl)methylene)propane-2-sulfinamide (233 mg). LC/MS ESI (m/z): 293 [M+H]⁺; ¹H NMR (400 MHz, DMSO-d6) δ 9.36 (s, 1H), 8.64 (s, 1H), 8.54 (s, 1H), 8.40 (s, 1H), 4.57 (t, J=6.9 Hz, 2H), 1.94-1.89 (m, 2H), 1.22 (s, 9H), 0.84 (t, J=7.4 Hz, 3H).

### Synthesis of (R)-2-methyl-N—((R)-1-(1-propyl-1H-pyrazolo[3,4-c]pyridin-5-yl)ethyl)propane-2-sulfinamide

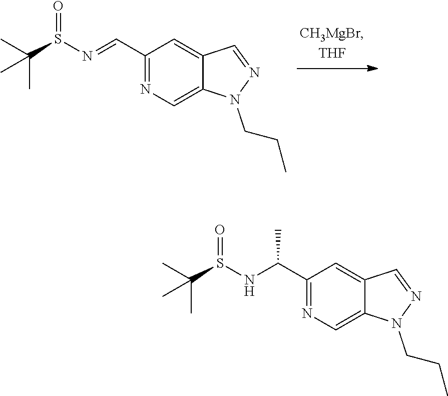

To a solution of (R,E)-2-methyl-N-((1-propyl-1H-pyrazolo[3,4-c]pyridin-5-yl)methylene)propane-2-sulfinamide (233 mg) in anhydrous THF (10 mL) was added 3M CH₃MgBr (1.328 mL, 3M in ether) dropwise under N₂, and the reaction was stirred at −60° C. for 2 hr. The reaction was quenched with aq. NH₄Cl and extracted with EA. The combined extracts were washed with saturated NaCl, dried over with Na₂SO₄, filtered and concentrated in vacuo. The residue was then purified by silica gel column chromatography to give (R)-2-methyl-N—((R)-1-(1-propyl-1H-pyrazolo[3,4-c]pyridin-5-yl)ethyl)propane-2-sulfinamide (146 mg). LC/MS ESI (m/z): 309 [M+H]⁺; ¹H NMR (400 MHz, DMSO-d6) δ 9.14 (s, 1H), 8.19 (d, J=0.6 Hz, 1H), 7.80 (d, J=0.6 Hz, 1H), 5.63 (d, J=7.5 Hz, 1H), 4.62-4.44 (m, 2H), 1.93-1.84 (m, 2H), 1.48 (d, J=6.8 Hz, 3H), 1.14 (s, 9H), 0.83 (t, J=7.4 Hz, 3H) ppm.

### Synthesis of (R)-1-(1-propyl-1H-pyrazolo[3,4-c]pyridin-5-yl)ethanamine, hydrochloride

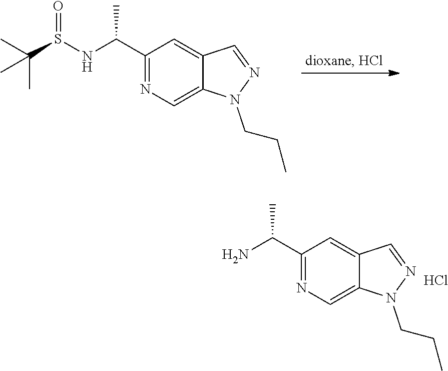

To a solution of (R)-2-methyl-N—((R)-1-(1-propyl-1H-pyrazolo[3,4-c]pyridin-5-yl)ethyl)propane-2-sulfinamide (95 mg) in dioxane (3 mL) was added 4N HCl-dioxane (1.0 mL). The reaction was stirred at rt for 30 min. Then the mixture was concentrated to provide (R)-1-(1-propyl-1H-pyrazolo[3,4-c]pyridin-5-yl)ethanamine hydrochloride, which was used directly in the next step. LC/MS ESI (m/z): 205 [M+H]⁺.

### Synthesis of (R)-2-(4-isopropylphenyl)-N-(1-(1-propyl-1H-pyrazolo[3,4-c]pyridin-5-yl)ethyl)acetamide

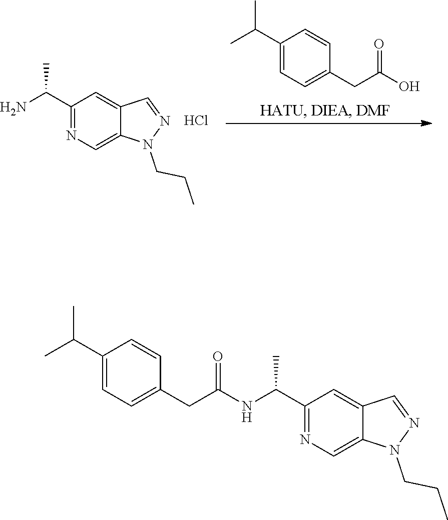 [SMILES: CCCn1ncc2cc([C@@H](C)N)ncc21 ; Cl ; CC(C)c1ccc(CC(=O)O)cc1 ; CCCn1ncc2cc([C@@H](C)NC(=O)Cc3ccc(C(C)C)cc3)ncc21]

To a solution of (R)-1-(1-propyl-1H-pyrazolo[3,4-c]pyridin-5-yl)ethanamine hydrochloride (82 mg) in anhydrous DMF were added DIEA (0.12 mL, Solution A). To a solution of 2-(4-isopropylphenyl)acetic acid (78.7 mg) in anhydrous DMF were added HATU (167.9 mg) and stirred 3 min at RT (Solution B). Solution A was added to the solution B and stirred at RT for 1 h. The mixture was diluted with water, and the aqueous phase was extracted with DCM (10 mL×3). The combined extracts were dried over Na₂SO₄, filtered and concentrated to give a residue, which was purified by Prep-HPLC [Column: YMC Triart C18 250*20 mm I.D,5 um; Mobile phase: from 10% to 95% MeCN with H₂O (0.1% formic acid); flow rate: 14 mL/min; wavelength: 220 nm/254 nm] to give (R)-2-(4-isopropylphenyl)-N-(1-(1-propyl-1H-pyrazolo[3,4-c]pyridin-5-yl)ethyl)acetamide. LC/MS ESI (m/z): 365 [M+H]⁺; ¹H NMR (400 MHz, CDCl₃) δ 8.82 (s, 1H), 7.98 (s, 1H), 7.49 (s, 1H), 7.19 (s, 4H), 6.62 (d, J=7.8 Hz, 1H), 5.28-5.21 (m, 1H), 4.42 (t, J=7.0 Hz, 2H), 3.56-3.55 (m, 2H), 2.92-2.88 (m, 1H), 2.00-1.96 (m, 2H), 1.46 (d, J=6.8 Hz, 3H), 1.25 (d, J=6.9 Hz, 6H), 0.93 (t, J=7.4 Hz, 3H).

### Example 3. (R)—N-(1-(1-ethyl-1H-pyrazolo[3,4-c]pyridin-5-yl)ethyl)-2-(4-isopropylphenyl)acetamide

### Synthesis of 5-bromo-1H-pyrazolo[3,4-c]pyridine

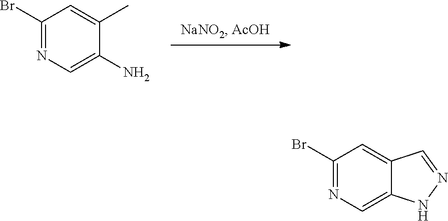

To a solution of 6-bromo-4-methylpyridin-3-amine (1.0 g) in acetic acid (60 mL) were added sodium nitrite (0.37 mL) in an ice bath. The reaction was stirred at RT overnight. Then the reaction was concentrated and then treated with saturated aqueous NaHCO₃. The aqueous mixture was extracted with EA. The combined extracts were washed with brine, dried over with Na₂SO₄, filtered and concentrated in vacuo. Then the residue was purified by silica gel column chromatography, to give 5-bromo-1H-pyrazolo[3,4-c]pyridine (442 mg). LC/MS ESI (m/z): 198 [M+H]⁺.

### Synthesis of 1H-pyrazolo[3,4-c]pyridine-5-carbaldehyde

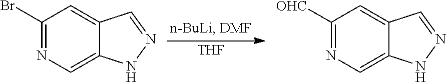 [SMILES: Brc1cc2cn[nH]c2cn1 ; O=Cc1cc2cn[nH]c2cn1 ; "THF"]

To a solution of 5-bromo-1H-pyrazolo[3,4-c]pyridine (3.3 g) in anhydrous THF (100 mL) was added n-BuLi (20.0 mL, 2 N in THF) at −78° C. The mixture was stirred at −78° C. for 1 hr. DMF (3.87 mL) was then added to the mixture at −78° C., and the mixture was stirred at −78° C. for 1 hr. The reaction was warmed to rt and quenched with an NH₄Cl solution. The two phases were separated, and the aqueous phase was extracted with DCM (10 mL×3). The combined organic layers were dried over Na₂SO₄, filtered and concentrated to give a residue, which was purified by column chromatography on silica gel (DCM:MeOH=1:0 to 20:1) to give 1H-pyrazolo[3,4-c]pyridine-5-carbaldehyde (1.0 g) as a white solid. LC/MS ESI (m/z): 148 [M+H]⁺

### Synthesis of 1-ethyl-1H-pyrazolo[3,4-c]pyridine-5-carbaldehyde

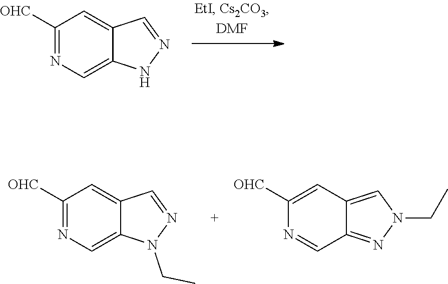

A solution of 1H-pyrazolo[3,4-c]pyridine-5-carbaldehyde (480 mg), ethyl iodide (0.35 mL) and Cs₂CO₃ (1.6 g) in DMF (1.0 mL) was stirred at 25° C. for 12 hr. The mixture was then diluted with water and extracted with EtOAc. The combined extracts were dried, filtered and concentrated to give a residue, which was purified by column chromatography on silica gel (DCM:MeOH=1:0 to 20:1) to give 1-ethyl-1H-pyrazolo[3,4-c]pyridine-5-carbaldehyde (185 mg) as a white solid. ¹H NMR (400 MHz, DMSO-d6) δ10.11 (s, 1H), 9.39 (s, 1H), 8.53-8.39 (m, 2H), 4.65 (q, J=7.2 Hz, 2H), 1.47 (t, J=7.2 Hz, 3H). and 2-propyl-2H-pyrazolo[3,4-c]pyridine-5-carbaldehyde (125 mg) as a white solid. ¹H NMR (400 MHz, DMSO): 10.05 (s, 1H), 9.36-9.23 (m, 1H), 8.85 (s, 1H), 8.43 (d, J=1.2 Hz, 1H), 4.60 (q, J=7.2 Hz, 2H), 1.55 (t, J=7.2 Hz, 3H).

### Synthesis of (R,E)-N-((1-ethyl-1H-pyrazolo[3,4-c]pyridin-5-yl)methylene)-2-methylpropane-2-sulfinamide

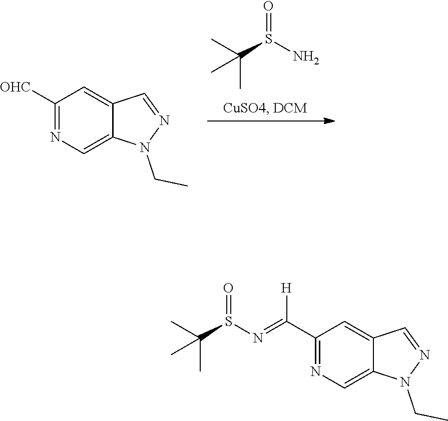

To a solution of 1-ethyl-1H-pyrazolo[3,4-c]pyridine-5-carbaldehyde (186 mg) in anhydrous DCM (10 mL) were added 2-methylpropane-2-sulfinamide (157.6 mg) and CuSO₄ (239.4 mg). The reaction was stirred at rt overnight. The mixture was then filtered, and the filtrate was concentrated in vacuum to get a residue, which was purified by silica to give (R,E)-N-((1-ethyl-1H-pyrazolo[3,4-c]pyridin-5-yl)methylene)-2-methylpropane-2-sulfinamide (207 mg) as a white solid. LC/MS ESI (m/z): 279 [M+H]⁺; ¹H NMR (400 MHz, DMSO-d6) δ 9.35 (s, 1H), 8.64 (s, 1H), 8.54 (d, J=1.2 Hz, 1H), 8.39 (d, J=0.6 Hz, 1H), 4.66-4.61 (m, 2H), 1.47 (t, J=7.2 Hz, 3H), 1.22 (s, 9H) ppm.

### Synthesis of (R)—N—((R)-1-(1-ethyl-1H-pyrazolo[3,4-c]pyridin-5-yl)ethyl)-2-methylpropane-2-sulfinamide

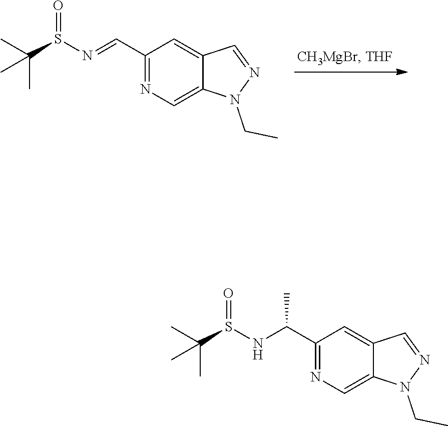

To a solution of (R,E)-N-((1-ethyl-1H-pyrazolo[3,4-c]pyridin-5-yl)methylene)-2-methylpropane-2-sulfinamide (207 mg) in anhydrous THF (15 mL) were added CH₃MgBr (1.24 mL, 3.0M in ether) dropwise. The reaction was stirred at −60° C. for 2 hr. The reaction was then quenched with saturated NH₄Cl solution. The two phases were separated, and the aqueous phase was extracted with DCM (10 mL×3). The combined organic layers were dried over Na₂SO₄, filtered and concentrated to give a residue, which was purified by silica gel column chromatography, to give (R)—N—((R)-1-(1-ethyl-1H-pyrazolo[3,4-c]pyridin-5-yl)ethyl)-2-methylpropane-2-sulfinamide (103 mg) as a yellow oil: LC/MS ESI (m/z): 295 [M+H]⁺.

### Synthesis of (R)-1-(1-ethyl-1H-pyrazolo[3,4-c]pyridin-5-yl)ethan-1-amine, hydrochloride

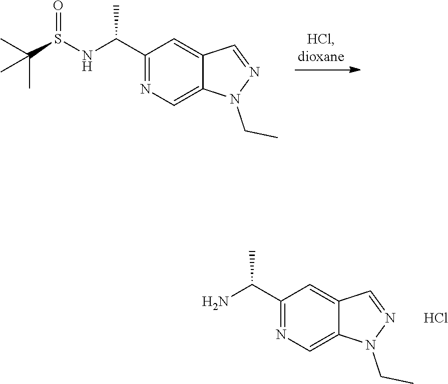 [SMILES: CCn1ncc2cc([C@@H](C)N[S@](=O)C(C)(C)C)ncc21 ; CCn1ncc2cc([C@@H](C)N)ncc21 ; Cl]

To a solution of (R)—N—((R)-1-(1-ethyl-1H-pyrazolo[3,4-c]pyridin-5-yl)ethyl)-2-methylpropane-2-sulfinamide (142 mg) in dioxane (3 mL) was added 4N HCl-dioxane (1 mL), and the reaction was stirred at rt for 30 min. The mixture was then concentrated to provide the (R)-1-(1-ethyl-1H-pyrazolo[3,4-c]pyridin-5-yl)ethan-1-amine, hydrochloride, which was used directly for next step. LC/MS ESI (m/z): 191 [M+H]⁺.

### Synthesis of (R)—N-(1-(1-ethyl-1H-pyrazolo[3,4-c]pyridin-5-yl)ethyl)-2-(4-isopropylphenyl)acetamide

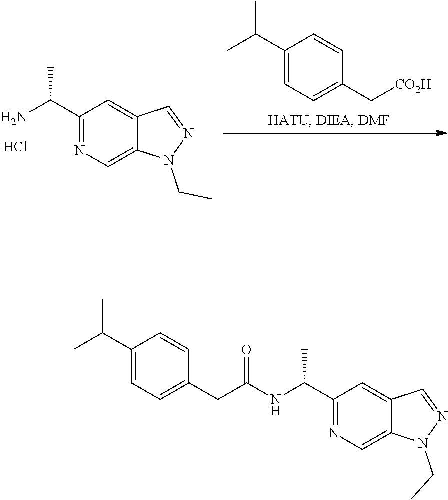 [SMILES: Cl ; CCn1ncc2cc([C@@H](C)N)ncc21 ; CC(C)c1ccc(CC(=O)O)cc1 ; CCn1ncc2cc([C@@H](C)NC(=O)Cc3ccc(C(C)C)cc3)ncc21]

To a solution of (R)-1-(1-ethyl-1H-pyrazolo[3,4-c]pyridin-5-yl)ethanamine hydrochloride (134 mg) in anhydrous DMF were added DIEA (0.5 mL) to provide Solution A. To a solution of 2-(4-isopropylphenyl)acetic acid (95.9 mg) in anhydrous DMF was added HATU (225 mg) and stirred at RT for 3 min to provide Solution B. Solution A was added to the Solution B and stirred at RT for 1 h. The reaction was diluted with EA and water. The two phases were separated, and the aqueous phase was extracted with DCM (10 mL×3). The combined organic layers were dried over Na₂SO₄, filtered and concentrated to give a residue, which was purified by prep-HPLC [Column: Xbudge prep C18 250*19 mm 5 um OBD; Mobile phase: from 10% to 95% MeCN with H₂O (0.1% formic acid); flow rate: 20 mL/min; wavelength: 205 nm/254 nm] and SFC (Column:ChiralPak IA, 250×21.3 mm I.D., 5 μm; Mobile phase: A for CO₂ and B for MEOH+0.1% NH3H2O; Gradient: B 40%; wavelength: 220 nm) to give (R)—N-(1-(1-ethyl-1H-pyrazolo[3,4-c]pyridin-5-yl)ethyl)-2-(4-isopropylphenyl)acetamide (44.8 mg) as a white solid. LC/MS ESI (m/z): 351 [M+H]⁺; ¹H NMR (400 MHz, CDCl₃) δ 8.83 (s, 1H), 7.97 (s, 1H), 7.49 (s, 1H), 7.19 (s, 4H), 6.62 (d, J=7.2 Hz, 1H), 5.28-5.21 (m, 1H), 4.52 (q, J=7.3 Hz, 2H), 3.55 (s, 2H), 2.92-2.87 (m, 1H), 1.56 (t, J=7.3 Hz, 3H), 1.46 (d, J=6.8 Hz, 3H), 1.25 (d, J=6.9 Hz, 6H).

### Example 4. (R)—N-(1-(1-ethyl-1H-pyrazolo[3,4-c]pyridin-5-yl)ethyl)-2-(4-isopropylphenyl)acetamide

### Step 1. Synthesis of 1-(2,2,2-trifluoroethyl)-1H-pyrazolo[3,4-c]pyridine-5-carbaldehyde

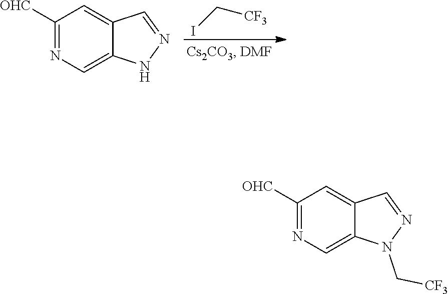

A solution of 1H-pyrazolo[3,4-c]pyridine-5-carbaldehyde (760 mg), 1,1,1-trifluoro-2-iodoethane (0.51 mL) and Cs₂CO₃ (2.5 g) in DMF (10 mL) was stirred at 100° C. for 12 hr. The mixture was diluted with water and extracted with EtOAc. The combined extracts were dried, filtered and concentrated to give a residue. The residue was purified by column chromatography on silica gel (DCM:MeOH=1:0 to 20:1) to give 1-(2,2,2-trifluoroethyl)-1H-pyrazolo[3,4-c]pyridine-5-carbaldehyde (500 mg) as a yellow solid. LC/MS ESI (m/z): 230 [M+H]⁺.

### Step 2. Synthesis of (R,E)-2-methyl-N-((1-(2,2,2-trifluoroethyl)-1H-pyrazolo[3,4-c]pyridin-5-yl)methylene)propane-2-sulfinamide

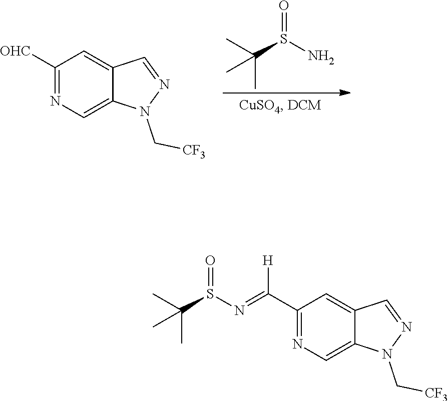

A solution of 1-(2,2,2-trifluoroethyl)-1H-pyrazolo[3,4-c]pyridine-5-carbaldehyde (500 mg), (R)-2-methylpropane-2-sulfinamide (396.7 mg) and copper sulfate pentahydrate (1.0 g) in DCM (15 mL) was stirred at 25° C. for 12 hr. The mixture was filtered and concentrated to give a residue. The residue was purified by column chromatography on silica gel (DCM:MeOH=1:0 to 20:1) to give (R,E)-2-methyl-N-((1-(2,2,2-trifluoroethyl)-1H-pyrazolo[3,4-c]pyridin-5-yl)methylene)propane-2-sulfinamide (450 mg) as a yellow solid. LC/MS ESI (m/z): 333 [M+H]⁺.

### Step 3. Synthesis of (R)-2-methyl-N—((R)-1-(1-(2,2,2-trifluoroethyl)-1H-pyrazolo[3,4-c]pyridin-5-yl)ethyl)propane-2-sulfinamide

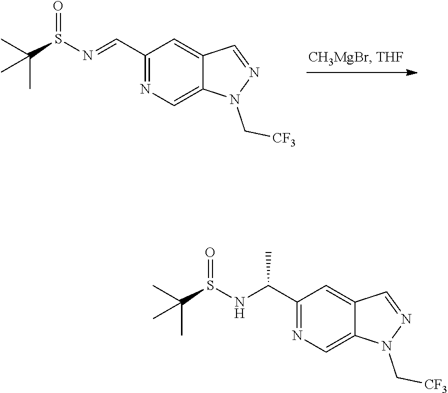 [SMILES: CC(C)(C)[S@@](=O)/N=C/c1cc2cnn(CC(F)(F)F)c2cn1 ; C[C@@H](N[S@](=O)C(C)(C)C)c1cc2cnn(CC(F)(F)F)c2cn1]

To a solution of (R,E)-2-methyl-N-((1-(2,2,2-trifluoroethyl)-1H-pyrazolo[3,4-c]pyridin-5-yl)methylene)propane-2-sulfinamide (450 mg) in THF (5 mL) was added CH₃MgBr (3.0M in ether, 1.35 mL) at −78° C. The mixture was stirred at −78° C. for 2 hr. The mixture was quenched with NH₄Cl solution and extracted with EtOAc. The combined extracts were dried, filtered and concentrated to give a residue. The residue was purified by column chromatography on silica gel (DCM:MeOH=1:0 to 20:1) to give (R)-2-methyl-N—((R)-1-(1-(2,2,2-trifluoroethyl)-1H-pyrazolo[3,4-c]pyridin-5-yl)ethyl)propane-2-sulfinamide (350 mg) as a yellow solid. LC/MS ESI (m/z): 349 [M+H]⁺.

### Step 4. Synthesis of (R)-1-(1-(2,2,2-trifluoroethyl)-1H-pyrazolo[3,4-c]pyridin-5-yl)ethan-1-amine hydrochloride

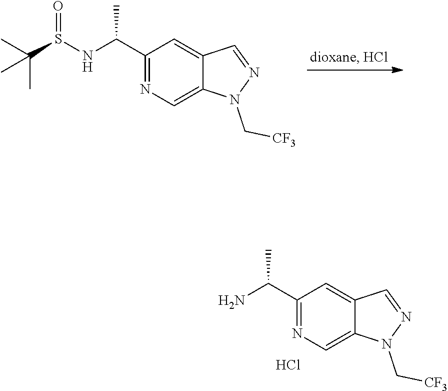 [SMILES: C[C@@H](N[S@](=O)C(C)(C)C)c1cc2cnn(CC(F)(F)F)c2cn1 ; C[C@@H](N)c1cc2cnn(CC(F)(F)F)c2cn1 ; Cl]

To a solution of (R)-2-methyl-N—((R)-1-(1-(2,2,2-trifluoroethyl)-1H-pyrazolo[3,4-c]pyridin-5-yl)ethyl)propane-2-sulfinamide (300 mg) in dioxane (2.0 mL) was added HCl/dioxane (4N in dioxane, 2.0 mL). The mixture was stirred at 25° C. for 30 min. The mixture was concentrated to give (R)-1-(1-(2,2,2-trifluoroethyl)-1H-pyrazolo[3,4-c]pyridin-5-yl)ethan-1-amine hydrochloride (300 mg, crude) as a white solid. LC/MS ESI (m/z): 245 [M+H]⁺.

### Step 5. Synthesis of (R)-2-(4-isopropylphenyl)-N-(1-(1-(2,2,2-trifluoroethyl)-1H-pyrazolo[3,4-c]pyridin-5-yl)ethyl)acetamide

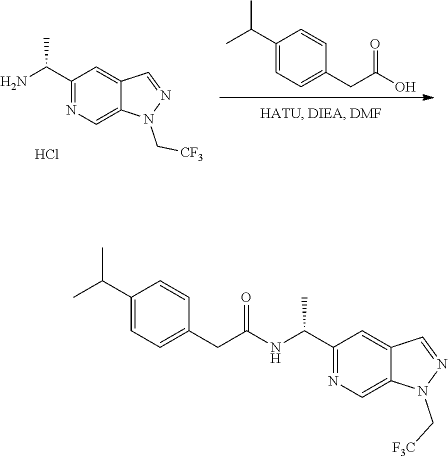 [SMILES: C[C@@H](N)c1cc2cnn(CC(F)(F)F)c2cn1 ; CC(C)c1ccc(CC(=O)O)cc1 ; Cl ; CC(C)c1ccc(CC(=O)N[C@H](C)c2cc3cnn(CC(F)(F)F)c3cn2)cc1]

A solution of (R)-1-(1-(2,2,2-trifluoroethyl)-1H-pyrazolo[3,4-c]pyridin-5-yl)ethan-1-amine hydrochloride (300 mg), 2-[4-(propan-2-yl)phenyl]acetic acid (240.8 mg), HATU (560.49 mg) and DIEA (0.41 mL) in DCM (10 mL) was stirred at 25° C. for 12 hr. The mixture was washed with water. The organic layer was dried, filtered and concentrated to give a residue. The residue was purified by prep-HPLC [Column: YMC-Actus Triart C18 250*21 mm; Mobile phase: from 50% to 95% MeCN with H2O (0.1% formic acid); flow rate: 20 mL/min; wavelength: 220 nm/254 nm] and SFC (Column:ChiralPak IA, 250×21.3 mm I.D., 5 μm; Mobile phase: A for CO₂ and B for MEOH+0.1% NH₃H₂O; Gradient: B 40%; wavelength: 220 nm) to give (R)-2-(4-isopropylphenyl)-N-(1-(1-(2,2,2-trifluoroethyl)-1H-pyrazolo[3,4-c]pyridin-5-yl)ethyl)acetamide (128.6 mg) as a white solid. LC/MS ESI (m/z): 405 (M+H)+; ¹H NMR (400 MHz, CDCl₃) δ 8.85 (s, 1H), 8.08 (s, 1H), 7.54 (s, 1H), 7.24-7.12 (m, 4H), 6.58 (d, J=7.6 Hz, 1H), 5.27-5.24 (m, 1H), 5.03 (q, J=8.4 Hz, 2H), 3.59-3.51 (m, 2H), 2.95-2.85 (m, 1H), 1.46 (d, J=6.8 Hz, 3H), 1.25 (d, J=6.6 Hz, 3H); ¹⁹F NMR (377 MHz, CDCl₃) δ −70.8 ppm.

### Example 5. Synthesis of 7-(4-ethylpiperazin-1-yl)-3H-phenothiazin-3-one (Compound 5)

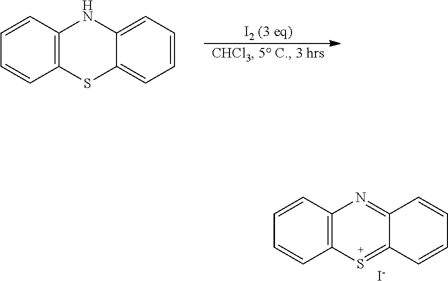

To a mixture of 10H-phenothiazine (15 g, 75.27 mmol) in CHCl₃ (750 mL) was added dropwise a mixture of I₂ (57 g, 226 mmol) in CHCl₃ (1.5 L) at 5° C. for 1 hr. The mixture was stirred at 5° C. for 3 hrs. The mixture was filtered and the filter cake was washed with methyl tert-butyl ether (500 mL). The filter cake was dried under vacuum to give crude phenothiazin-5-ium iodide (15 g) as a dark blue solid: ¹H NMR (400 MHz, DMSO) δ 7.38-7.91 (m, 3H), 7.93-8.42 (m, 4H), 8.45-8.70 (m, 1H).

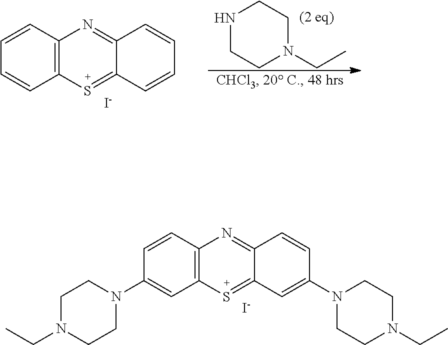

To a mixture of phenothiazin-5-ium iodide (15 g, 46.13 mmol) in CHCl₃ (300 mL) was added 1-ethylpiperazine (10.54 g, 92.26 mmol) at 20° C. The mixture was stirred at 20° C. for 48 hrs. The mixture was concentrated under vacuum to give a residue. The residue was washed with methyl tert-butyl ether (100 mL) and dried under vacuum to give crude 3,7-bis(4-ethylpiperazin-1-yl)phenothiazin-5-ium iodide (15 g) as a blue solid. LC/MS conditions: the column used for chromatography was a ZORBAX Eclipse XDB-C18 2.1*30 mm, (3.5 um particles). Detection methods were diode array (DAD). MS mode was positive electrospray ionization. MS range was 100-1000. Mobile phase A was 0.037% Trifluoroacetic acid in water, and mobile phase B was 0.018% Trifluoroacetic acid in HPLC grade acetonitrile. The gradient was 5-95% B in 2.20 min 0.5% B in 0.01 min, 5-95% B (0.01-1.00 min), 95-100% B (1.00-1.80 min), 5% B in 1.81 min with a hold at 5% B for 0.39 min. The flow rate was 1.0 mL/min. LCMS (ESI+): 0.373 min, m/z 422.2.

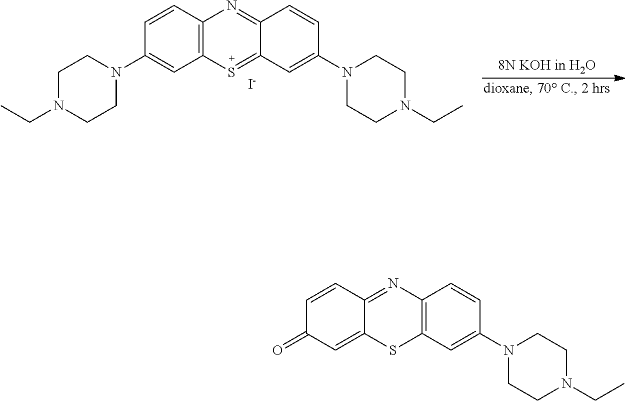

To a mixture of crude 3,7-bis(4-ethylpiperazin-1-yl)phenothiazin-5-ium iodide (5 g, 9.1 mmol) in dioxane (30 mL) was added KOH (8 N, 28.5 mL) at 20° C. The mixture was stirred at 70° C. for 2 hrs. The mixture was cooled to 15° C. and concentrated under vacuum to give a residue. The residue was extracted with ethyl acetate (100 mL). The organic layer was concentrated under vacuum to give crude product. The crude product was purified via column chromatography on silica gel (ethyl acetate:methanol=5:1) three times, and then purified by normal-phase liquid chromatography and lyophilized to give 7-(4-ethylpiperazin-1-yl)-3H-phenothiazin-3-one (Compound 5, 55 mg) as a brown solid: ¹H NMR (400 MHz, CDCl₃) δ 1.17 (br t, J=7.23 Hz, 3H), 2.52 (br d, J=6.80 Hz, 2H), 2.64 (br s, 4H), 3.51 (br d, J=5.04 Hz, 4H), 6.70 (d, J=2.19 Hz, 1H), 6.79 (d, J=2.85 Hz, 1H), 6.86 (dd, J=9.87, 2.19 Hz, 1H), 7.02 (dd, J=9.21, 2.63 Hz, 1H), 7.57 (d, J=9.87 Hz, 1H), 7.73 (d, J=9.21 Hz, 1H).

### Example 6. Synthesis of Bicyclic Compounds of Formula II

HPLCMS Method A: The column was an Xbridge Shield RP18 2.1*50 mm, (5 um particles). UV detection was by diode array (DAD). The MS mode was positive electrospray ionization. The MS range was 100-1000. Mobile phase A was 10 mM ammonium bicarbonate in water. Mobile phase B was HPLC grade acetonitrile. The gradient was 5-95% B in 2.05 min. The flow rate was 1.0 mL/min.

HPLCMS Method B: The column was a ZORBAX Eclipse XDB-C18 2.1*30 mm, (3.5 um particles). UV detection was by diode array (DAD). The MS mode was positive electrospray ionization. The MS range was 100-1000. Mobile phase A was 0.037% trifluoroacetic acid in water. Mobile phase B was 0.018% trifluoroacetic acid in HPLC grade acetonitrile. The gradient was 5-95% B in 2.20 min. The flow rate was 1.0 mL/min.

Preparative HPLC Method: The column was a GromSil 80 Si NP-1 5 μm, 250 mm×4.6 mm column. The mobile phase was heptane/ethanol with a gradient of 5%-95% ethanol over 15 min.

### Synthesis of Intermediate 8-ethyl-3,8-diazabicyclo[3.2.1]octane (INT-2)

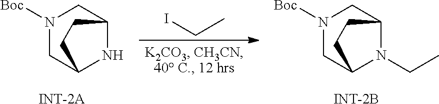

To a mixture of INT-2A (20 g, 94.21 mmol) and K₂CO₃ (26.04 g, 188.42 mmol) in acetonitrile (200 mL) was added ethyl iodide (14.69 g, 94.21 mmol) at 15° C. The mixture was stirred at 40° C. for 12 hours under N₂. LCMS indicated starting material was consumed, and desired product was detected. The reaction mixture was cooled to 15° C. The mixture was filtered, and the filtrate was concentrated under reduced pressure. The residue was purified by column chromatography on silica gel (ethyl acetate) to give INT-2B (14 g, 58.25 mmol, 62% yield) as a white solid. HPLCMS Method A (ESI+): m/z 241.2 (MH+), RT 0.765 min.

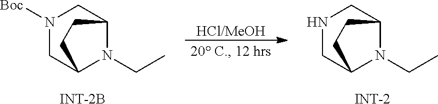

To a mixture of INT-2B (14 g, 58.25 mmol) in methanol (100 mL) was added HC/MeOH (4 M, 87.50 mL) at 20° C. The mixture was stirred at 20° C. for 12 hrs. The reaction mixture was concentrated under reduced pressure to give crude 8-ethyl-3,8-diazabicyclo[3.2.1]octane (INT-2) dihydrochloride (10 g, 46.92 mmol, 80% yield) as a yellow solid.

### Synthesis of Intermediate 3-ethyl-3,8-diazabicyclo[3.2.1]octane (INT-3)

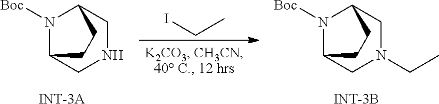

To a mixture of INT-3A (20 g, 94.21 mmol) and K₂CO₃ (26.04 g, 188.42 mmol) in acetonitrile (200 mL) was added ethyl iodide (14.69 g, 94.21 mmol) at 15° C. The mixture was stirred at 40° C. for 12 hours under N₂. LCMS indicated starting material was consumed, and desired product was detected. The reaction mixture was cooled to 15° C. The mixture was filtered, and the filtrate was concentrated under reduced pressure, and the residue was purified by column chromatography on silica gel (ethyl acetate) to give INT-3B (14 g, 58.25 mmol, 62% yield) as a white solid. HPLCMS Method A (ESI+): m/z 241.2 (MH+), RT 0.853 min.

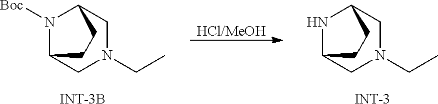

To a mixture of INT-3B (14 g, 58.25 mmol) in methanol (100 mL) was added HC/MeOH (4 M, 87.50 mL) at 20° C. The mixture was stirred at 20° C. for 12 hrs. The reaction mixture was concentrated under reduced pressure to give crude 3-ethyl-3,8-diazabicyclo[3.2.1]octane (INT-3) dihydrochloride (10 g, 46.92 mmol, 80% yield) as a yellow solid.

### Synthesis of 7-((1R,5S)-3-ethyl-3,8-diazabicyclo[3.2.1]octan-8-yl)-3H-phenothiazin-3-one (Compound 6)

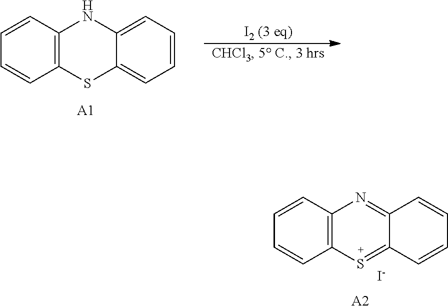

To a mixture of A1 (10 g, 50.18 mmol) in CHCl₃ (500 mL) was added dropwise a mixture of iodine (38.21 g, 150 mmol) in CHCl₃ (750 mL) at 5° C. for 1 hr. The mixture was stirred at 5° C. for 3 hours. The mixture was filtered, and the filter cake was washed with CHCl₃ (200 mL). The filter cake was dried by high vacuum to give crude A2 (10 g) as a dark blue solid. ¹H NMR (400 MHz, (CD₃)₂SO) δ 7.30-7.76 (m, 3H), 7.85-8.19 (m, 4H), 8.36-8.63 (m, 1H).

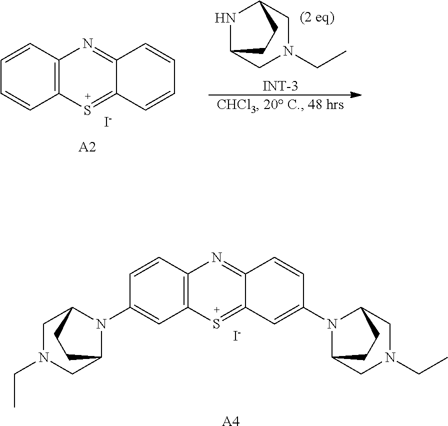

To a mixture of A2 (7.6 g, 23.37 mmol) and 3-ethyl-3,8-diazabicyclo[3.2.1]octane dihydrochloride (9.96 g, 46.75 mmol, 2 eq) in CHCl₃ (450 mL) was added N-ethyl-N-isopropylpropan-2-amine (15.10 g, 116.86 mmol, 5 eq) at 20° C. The mixture was stirred at 20° C. for 48 hrs. The mixture was concentrated under reduced pressure, and the residue was washed with methyl tert-butyl ether (100 mL) and dried by high vacuum to give crude A4 (9 g) as a blue solid. HPLCMS Method B (ESI+): m/z 474.3 (M+), RT 0.176 min.

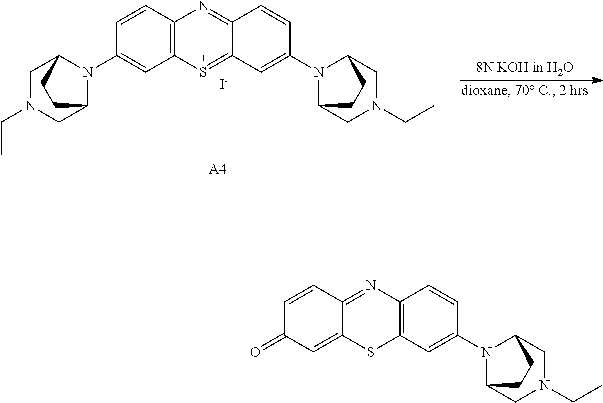

To a mixture of crude A4 (9 g, 14.96 mmol) in dioxane (150 mL) was added potassium hydroxide (8 N, 150 mL) at 20° C. The mixture was stirred at 70° C. for 2 hrs. The mixture was cooled to 15° C. and concentrated under reduced pressure, and the residue was extracted with ethyl acetate (300 mL). The organic layer was concentrated under reduced pressure, and the crude product was purified by column chromatography on silica gel (ethyl acetate:methanol=1:1) three times, and then purified twice by normal-phase preparative HPLC to give after lyophilization Compound 6 (55 mg) as a brown solid. ¹H NMR (400 MHz, CDCl₃) δ 1.02 (t, J=7.28 Hz, 3H), 1.98-2.03 (m, 2H), 2.07-2.15 (m, 2H), 2.30-2.41 (m, 4H), 2.72 (dd, J=10.79, 2.26 Hz, 2H), 4.34 (br d, J=2.01 Hz, 2H), 6.67 (dd, J=12.80, 2.76 Hz, 2H), 6.85 (ddd, J=11.54, 9.29, 2.26 Hz, 2H), 7.56 (d, J=10.04 Hz, 1H), 7.69 (d, J=9.03 Hz, 1H). HPLCMS Method A (ESI+): m/z 352.1 (MH+), RT 2.621 min.

### Synthesis of 7-((1R,5S)-8-ethyl-3,8-diazabicyclo[3.2.1]octan-3-yl)-3H-phenothiazin-3-one (Compound 7)

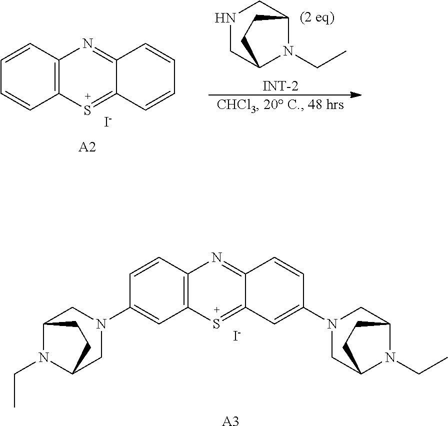

To a mixture of A2 (7.6 g, 23.37 mmol) and 8-ethyl-3,8-diazabicyclo[3.2.1]octane dihydrochloride (9.96 g, 46.75 mmol, 2 eq) in CHCl₃ (450 mL) was added N-ethyl-N-isopropylpropan-2-amine (15.10 g, 116.86 mmol, 5 eq) at 20° C. The mixture was stirred at 20° C. for 48 hours. The mixture was concentrated under reduced pressure, and the residue was washed with methyl tert-butyl ether (100 mL) and dried by high vacuum to give crude A3 (9 g) as a blue solid. HPLCMS Method B (ESI+): m/z 474.3 (M+), RT 0.461 min.

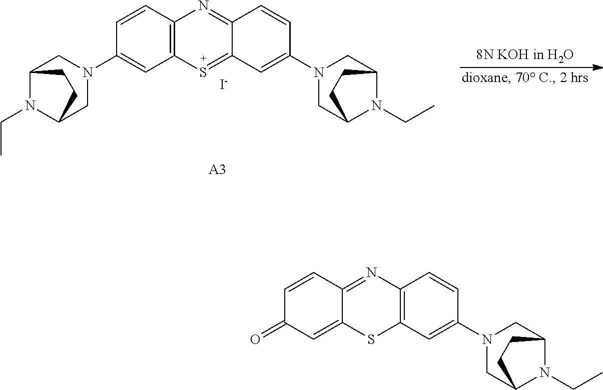

To a mixture of crude A3 (9 g, 14.96 mmol) in dioxane (150 mL) was added potassium hydroxide (8 N, 150 mL) at 20° C. The mixture was stirred at 70° C. for 2 hrs. The mixture was cooled to 15° C. and concentrated under reduced pressure to remove most of the organic solvents. The residue was extracted with ethyl acetate (300 mL). The organic layer was concentrated under reduced pressure, and the crude product was purified by column chromatography on silica gel (from ethyl acetate to ethyl acetate:methanol=1: 1) three times, and then purified twice by normal-phase preparative HPLC to give after lyophilization Compound 7 (60 mg) as a brown solid. ¹H NMR (400 MHz, CDCl₃) δ 1.15 (t, J=7.28 Hz, 3H), 1.70 (d, J=7.53 Hz, 2H), 2.00-2.07 (m, 2H), 2.45-2.55 (m, 2H), 3.26 (dd, J=11.04, 2.01 Hz, 2H), 3.46 (br d, J=1.51 Hz, 2H), 3.49-3.55 (m, 2H), 6.68 (t, J=2.51 Hz, 2H), 6.84 (dd, J=9.79, 2.26 Hz, 1H), 6.92 (dd, J=9.03, 3.01 Hz, 1H), 7.55 (d, J=9.54 Hz, 1H), 7.70 (d, J=9.03 Hz, 1H). HPLCMS Method A (ESI+): m/z 352.1 (MH+), RT 2.352 min.

The following compounds were prepared by similar methods.

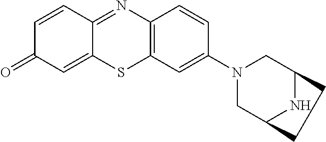 [SMILES: O=c1ccc2nc3ccc(N4C[C@H]5CC[C@@H](C4)N5)cc3sc-2c1]

Compound 8: ¹H NMR (400 MHz, (CD₃)₂SO) δ 1.70 (d, J=7.53 Hz, 2H), 2.00-2.07, 3.26 (dd, J=11.04, 2.01 Hz, 2H), 3.46 (br d, J=1.51 Hz, 2H), 3.49-3.55 (m, 2H), 6.68 (t, J=2.51 Hz, 2H), 6.84 (dd, J=9.79, 2.26 Hz, 1H), 6.92 (dd, J=9.03, 3.01 Hz, 1H), 7.55 (d, J=9.54 Hz, 1H), 7.70 (d, J=9.03 Hz, 1H). HPLCMS Method A (ESI+): m/z 324.1 (MH+), RT 1.852 min.

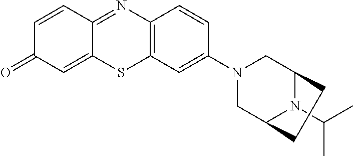 [SMILES: CC(C)N1[C@@H]2CC[C@H]1CN(c1ccc3nc4ccc(=O)cc-4sc3c1)C2]

Compound 9: ¹H NMR (400 MHz, CDCl₃) δ 1.15 (d, J=7.28 Hz, 6H), 1.70 (d, J=7.53 Hz, 2H), 2.00-2.07 (m, 2H), 2.60-2.70 (m, 1H), 3.26 (dd, J=11.04, 2.01 Hz, 2H), 3.46 (br d, J=1.51 Hz, 2H), 3.49-3.55 (m, 2H), 6.68 (t, J=2.51 Hz, 2H), 6.84 (dd, J=9.79, 2.26 Hz, 1H), 6.92 (dd, J=9.03, 3.01 Hz, 1H), 7.55 (d, J=9.54 Hz, 1H), 7.70 (d, J=9.03 Hz, 1H). HPLCMS Method A (ESI+): m/z 366.1 (MH+), RT 2.492 min.

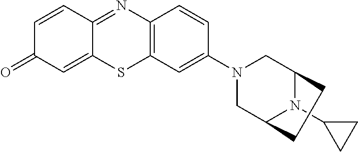 [SMILES: O=c1ccc2nc3ccc(N4C[C@H]5CC[C@@H](C4)N5C4CC4)cc3sc-2c1]

Compound 10: ¹H NMR (600 MHz, CD₂Cl₂) δ 7.66 (d, J=9.2 Hz, 1H), 7.51 (d, J=9.7 Hz, 1H), 6.93 (dd, J=2.8, 9.2 Hz, 1H), 6.74 (dd, J=2.1, 9.8 Hz, 1H), 6.72 (d, J=2.6 Hz, 1H), 6.61 (d, J=2.2 Hz, 1H), 3.55-3.47 (m, 4H), 3.16 (br d, J=10.6 Hz, 2H), 2.12-2.05 (m, 2H), 1.90 (br s, 1H), 1.71 (br d, J=7.6 Hz, 2H), 0.52-0.43 (m, 4H) HPLCMS Method A (ESI+): m/z 364.1 (MH+), RT 2.411 min.

### Example 7. Effects of Compound 1 in Stressed Dopaminergic Neurons

General: The rat primary midbrain neuron co-culture system was used to demonstrate rescue of dopamine neuron viability, dopamine neuron neurite length, Miro1 release, and pathological phospho-Serine129 alpha-synuclein (a biomarker associated with Lewy Body and Lewy Neurite pathologies associated with Parkinson's disease as well as other synuclein-related pathologies, including Multiple System Atrophy and Alzheimer's disease). The neuronal challenge was alpha-synuclein oligomers.

After incubation with alpha-synuclein (α-syn) oligomer, cells were fixed by a solution of 4% paraformaldehyde for 20 min at room temperature, the control conditions were fixed as well following the same procedure. The cells were then permeabilized and non-specific sites were blocked with a solution of phosphate buffered saline containing (PBS) with saponin and FCS for 15 min at room temperature. Cells were incubated with a chicken Anti-Tyrosine Hydroxylase antibody (TH, Abcam, ref 76442), a mouse monoclonal Anti-α-syn P129 antibody (Abcam, ref ab184674) and a Rabbit polyclonal Anti-Miro1 antibody (Sigma, ref: HPA010687) in PBS with saponin and FCS overnight at 4° C. These antibodies were revealed with an Alexa Fluor 568 goat anti-mouse IgG (Molecular probe), an Alexa Fluor 647 goat anti-chicken IgG (Molecular probe) and an Alexa Fluor 488 goat anti-rabbit IgG (Molecular probe) in PBS with 1% FCS, 0.1% saponin, for 1 h at room temperature. Nuclei of cells were labelled by a fluorescent marker (Hoechst solution) in the same solution.

For each well of culture, pictures were taken using InCell Analyzer™ 2200 (GE Healthcare) with 20× magnification. All the images were taken in the same conditions. The number of dopaminergic neurons (TH), the neurite length of dopaminergic neurons, the signal of phosphorylated 129 of α-syn in dopaminergic neurons and the signal of Miro1 in dopaminergic neurons were automatically evaluated with Developer system analysis (GE Healthcare). All values were expressed as mean±standard error of the mean. Statistical analyses were done on the different conditions (ANOVA followed by Dunnett's test).

The T-type calcium channel antagonist Compound 1 was described in PCT application PCT/US22/18682 (published as WO 2022/216386) as Example 15 having Cav3.2 IC₅₀=163 nM. Compound 1 was co-dosed with alpha-synuclein oligomers to determine if it could prevent and/or rescue neuronal damage caused by alpha-synuclein oligomer administration.

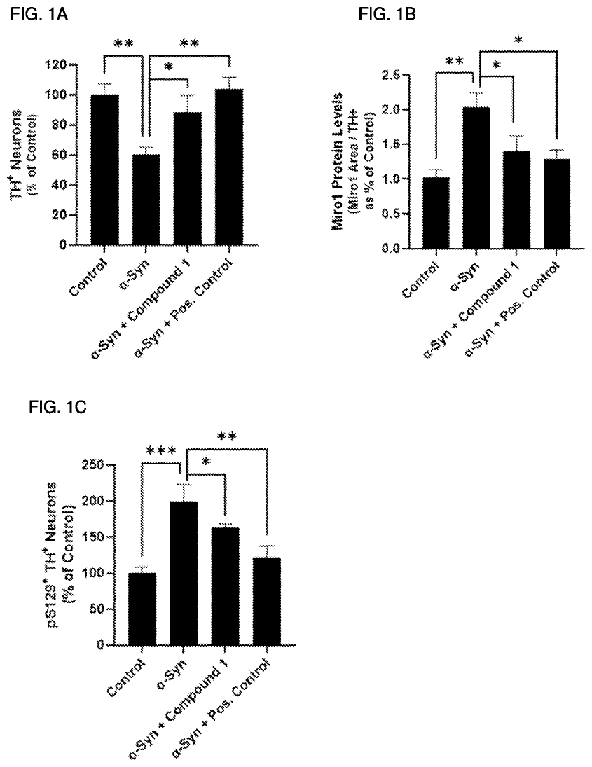

In an initial study, rat primary midbrain neuron co-cultures were incubated with alpha-synuclein oligomer alone, with 400 nM Compound 1, or with positive control. Compound 1 prevented TH+ neuronal loss induced by alpha-synuclein oligomer (FIG. 1A) at a statistically significant level. Compound 1 also exhibited statistically significant rescue of Miro1 release (FIG. 1B) and reduction of pathogenic phosphorylated serine-129 (pS129) alpha-synuclein levels (FIG. 1C), with EC₅₀˜250 nM for rescue of TH-positive neuron viability. Positive control also prevented TH+ neuronal loss, prevented Miro1 release, and reduced pS129 alpha-synuclein levels.

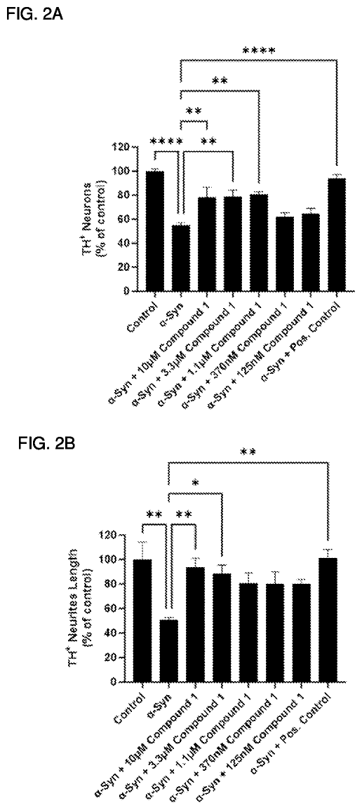

In a subsequent dose-response experiment, Compound 1 rescued dopamine neuron survival in a dose-dependent fashion (FIG. 2A). Additionally, Compound 1 rescued dopamine neuron neurite length in a dose-dependent fashion following alpha-synuclein (α-syn) oligomer challenge (FIG. 2B), with EC₅₀<150 nM for rescue of TH-positive neurite length. The assay positive control also rescued dopamine neuron loss and neurite length.

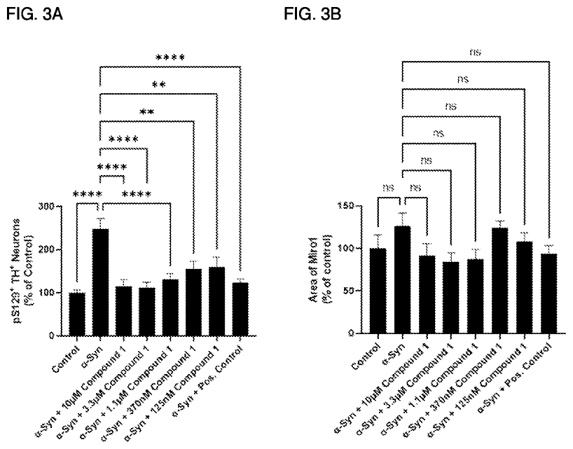

Compound 1 lowered levels of pathogenic pS129 α-synin a dose-dependent fashion (FIG. 3A), with IC₅₀<125 nM. Further, there was a trend towards rescue of Miro1 elevation due to alpha-synuclein oligomer challenge with Compound 1, but it did not reach statistical significance (FIG. 3B). Positive control also lowered levels of pS129 α-synin a statistically significant manner but did not significantly affect Miro1 levels as compared to aSyn challenge alone.

In the absence of any neuronal challenge, Compound 1 did not affect Miro1 levels, TH+ neuron count, TH+ neurite length, or number of pS129+ TH+ neurons in a statistically significant manner.

### Example 8. Effects of Compound 5 in Stressed Dopaminergic Neurons

### Ferroptosis Assay

Human fibroblast survival assay. All fibroblasts were obtained from the National Institute of Neurological Disorders and Stroke (NINDS) cell line repository housed at Rutgers University. Compounds were tested in at least one of the following Parkinson's disease subject-derived fibroblast lines: Line ND29802 (LRRK2 G2019S mutation carrier), Line ND40996 (SNCA A53T mutation carrier), or Line ND34263 (GBA N370S mutation carrier).

Human fibroblasts were seeded into 96-well plates on Day 0. On Day 1, fibroblasts were challenged with RSL3 (a ferroptosis activator, CAS No. 1219810-16-8) alone or in co-treatment with test compound administered in dose response. On Day 2, fibroblasts were fixed and stained by the nuclear stain 4′, 6-diamidino-2-phenylindole (DAPI), and the number of DAPI-positive nuclei were counted for each condition using the Yokogawa CQ1 imaging platform to quantify the EC₅₀ for cell survival.

Compound 5 was shown to be a ferroptosis inhibitor in the above assay with EC₅₀=3.6 nM.

General: The rat primary midbrain neuron co-culture system was used to demonstrate rescue of dopamine neuron viability, dopamine neuron neurite length, Miro1 release, and pathological phospho-Serine129 alpha-synuclein (a biomarker associated with Lewy Body and Lewy Neurite pathologies associated with Parkinson's disease as well as other synuclein-related pathologies, including Multiple System Atrophy and Alzheimer's disease). The neuronal challenge was alpha-synuclein oligomers.

After incubation with alpha-synuclein (α-syn) oligomer, cells were fixed by a solution of 4% paraformaldehyde for 20 min at room temperature, the control conditions were fixed as well following the same procedure. The cells were then permeabilized and non-specific sites were blocked with a solution of phosphate buffered saline containing (PBS) with saponin and FCS for 15 min at room temperature. Cells were incubated with a chicken Anti-Tyrosine Hydroxylase antibody (TH, Abcam, ref 76442), a mouse monoclonal Anti-α-syn P129 antibody (Abcam, ref ab184674) and a Rabbit polyclonal Anti-Miro1 antibody (Sigma, ref: HPA010687) in PBS with saponin and FCS overnight at 4° C. These antibodies were revealed with an Alexa Fluor 568 goat anti-mouse IgG (Molecular probe), an Alexa Fluor 647 goat anti-chicken IgG (Molecular probe) and an Alexa Fluor 488 goat anti-rabbit IgG (Molecular probe) in PBS with 1% FCS, 0.1% saponin, for 1 h at room temperature. Nuclei of cells were labelled by a fluorescent marker (Hoechst solution) in the same solution.

For each well of culture, pictures were taken using InCell Analyzer™ 2200 (GE Healthcare) with 20× magnification. All the images were taken in the same conditions. The number of dopaminergic neurons (TH), the neurite length of dopaminergic neurons, the signal of phosphorylated 129 of α-syn in dopaminergic neurons and the signal of Miro1 in dopaminergic neurons were automatically evaluated with Developer system analysis (GE Healthcare). All values were expressed as mean±standard error of the mean. Statistical analyses were done on the different conditions (ANOVA followed by Dunnett's test).

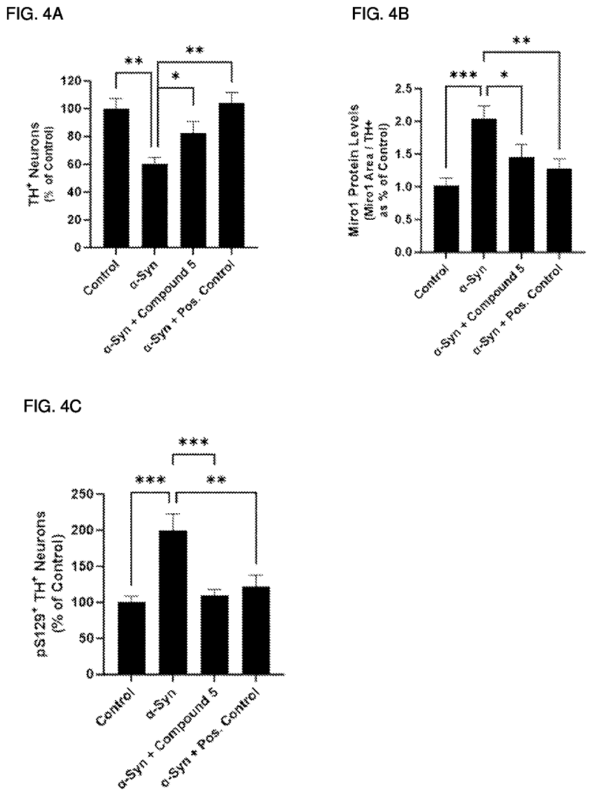

In an initial study, rat primary midbrain neuron co-cultures were incubated with alpha-synuclein oligomer alone, with 40 nM Compound 5, or with positive control. Compound 1 prevented TH+ neuronal loss induced by alpha-synuclein oligomer (FIG. 4A) at a statistically significant level. Compound 5 also exhibited statistically significant rescue of Miro1 release (FIG. 4B) and reduction of pathogenic phosphorylated serine-129 (pS129) alpha-synuclein levels (FIG. 4C), with EC₅₀˜7 nM for rescue of TH-positive neuron viability. Positive control also prevented TH+ neuronal loss, prevented Miro1 release, and reduced pS129 alpha-synuclein levels.

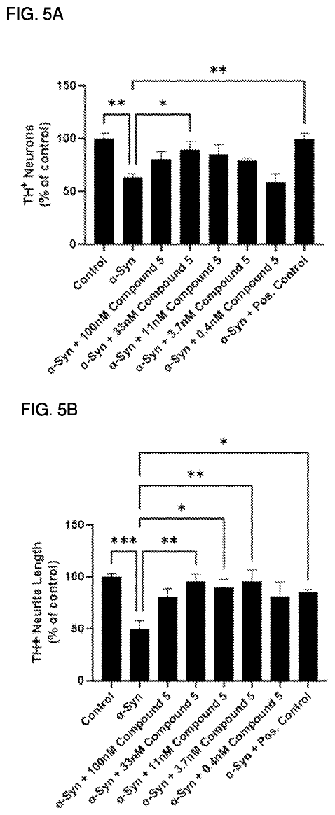

In a subsequent dose-response experiment, Compound 5 rescued dopamine neuron survival in a dose-dependent fashion (FIG. 5A). Additionally, Compound 5 rescued dopamine neuron neurite length in a dose-dependent fashion following alpha-synuclein (α-syn) oligomer challenge (FIG. 5B), with EC₅₀<0.4 nM for rescue of TH-positive neurite length. The assay positive control also rescued dopamine neuron loss and neurite length.

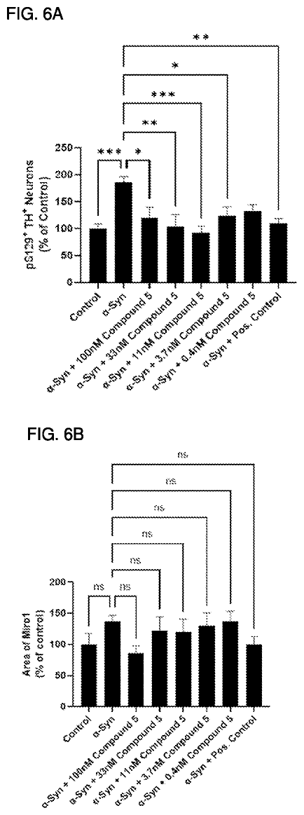

Compound 5 lowered levels of pathogenic pS129 α-synin a dose-dependent fashion (FIG. 6A), with IC₅₀<0.4 nM. Further, there was a trend towards rescue of Miro1 elevation due to alpha-synuclein oligomer challenge with Compound 5, but it did not reach statistical significance (FIG. 6B). Positive control also lowered levels of pS129 α-synin a statistically significant manner but did not significantly affect Miro1 levels as compared to α-synchallenge alone.

In the absence of any neuronal challenge, Compound 5 did not affect Miro1 levels, TH+ neuron count, TH+ neurite length, or number of pS129+TH+ neurons in a statistically significant manner.

### Example 9. Immunocytochemistry in Primary Rat Midbrain Neuron Co-Culture

Immunocytochemistry was performed for DAPI (nuclear dye), Tyrosine Hydroxylase (TH, a marker for dopaminergic neurons), and phospho-Serine129 alpha-synuclein (pS129 α-syn, a pathogenic form of alpha-synuclein found in Lewy Body pathology) in primary rat midbrain neuron co-cultures. In naive untreated neuronal cultures, healthy TH-positive dopaminergic neurons with elaborate neurites were observed and there was very low pS129 α-synbackground signal. When these neuronal cultures were stressed with toxic exogenous alpha-synuclein oligomers (500 nM, 96 hours), there was a large and statistically significant (1) loss of TH+dopaminergic neuron viability, (2) decrease of TH+dopaminergic neuron neurite length, (3) increase in pS129 α-syn-positive Lewy Body formation, and (4) increase in Miro1 protein levels. However, when these alpha-synuclein oligomer stressed neuron cultures were pre-treated with Compound 1 (400 nM) or Compound 5 (40 nM), there was a statistically significant (1) rescue of TH-positive dopaminergic neuron viability, (2) rescue of TH+dopaminergic neuron neurite length, (3) decrease/prevention in pS129 α-syn-positive Lewy Body formation, and (4) decrease in Miro1 protein levels. In addition, toxic exogenous alpha-synuclein oligomers caused pS129 α-syn-positive Lewy Body formation to occur in TH-negative non-dopaminergic neurons, and there was a robust decrease/prevention in pS129 α-syn-positive Lewy Body formation with pre-treatment of either Compound 1 (1.1 uM) or Compound 5 (3.7 nM).

### Example 10. Compound 1 Treatment in Primary Rat Dopaminergic Neurons after Alpha-Synuclein Oligomer Injury

Interventional treatment with Compound 1 removed pre-existing phospho-serine129 alpha-synuclein pathology in rat midbrain dopamine neuron co-cultures previously challenged with toxic alpha-synuclein oligomers.

Rat dopaminergic neurons were cultured as previously described (Schinelli, S. et al. Journal of Neurochemistry 1988, 50(6), pages 1900-1907). After 7 days of culture, cells were challenged with alpha-synuclein oligomer (500 nM) for 48 hours. Subsequent to the challenge, cells were subjected to alpha-synuclein (α-syn) oligomer (500 nM)+various concentrations of Compound 1 (0, 14, 41, 123, 370, 1100, 3300, and 10000 nM) for an additional 48 hours. By condition, N=6 wells were performed and one plate of 96 wells were performed.

After 4 days of incubation with α-syn oligomer, cells were fixed by a solution of 4% paraformaldehyde for 20 min at room temperature, the control conditions were fixed as well following the same procedure. The cells were then permeabilized and non-specific sites were blocked with a solution of phosphate buffered saline containing (PBS) with saponin and FCS for 15 min at room temperature. Cells were incubated with a chicken Anti-Tyrosine Hydroxylase antibody (TH, Abcam, ref 76442), a mouse monoclonal Anti-α-syn P129 antibody (Abcam, ref ab184674) and a Rabbit polyclonal Anti-Miro1 antibody (Sigma, ref: HPA010687) in PBS with saponin and FCS overnight at 4° C. These antibodies were revealed with an Alexa Fluor 568 goat anti-mouse IgG (Molecular probe), an Alexa Fluor 647 goat anti-chicken IgG (Molecular probe) and an Alexa Fluor 488 goat anti-rabbit IgG (Molecular probe) in PBS with 1% FCS, 0.1% saponin, for 1 h at room temperature. Nuclei of cells were labelled by a fluorescent marker (Hoechst solution) in the same solution.

For each well of culture, pictures were taken using InCell Analyzer™ 2200 (GE Healthcare) with 20× magnification. All the images were taken in the same conditions. The number of dopaminergic neurons (TH), the neurite length of dopaminergic neurons, the signal of phosphorylated 129 of α-syn in dopaminergic neurons and the signal of Miro1 in dopaminergic neurons were automatically evaluated with Developer system analysis (GE Healthcare). All values were expressed as mean±standard error of the mean. Statistical analyses were done on the different conditions (ANOVA followed by Dunnett's test).

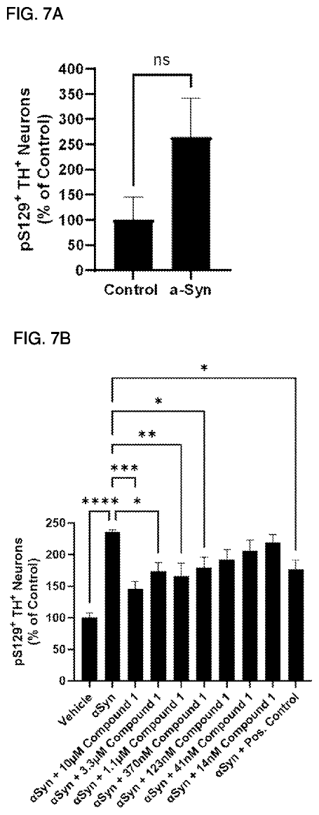

Cellular challenge with alpha-synuclein oligomer over 2 days afforded a trend toward higher levels of phosphorylated Ser129-positive TH+ neurons (FIG. 7A). After continued challenge with alpha-synuclein oligomer and adding treatment with Compound 1 over 2 more days, the levels of phosphorylated Ser129-positive TH+ neurons were reduced compared to no treatment (FIG. 7B). At completion of the study (4 days total), the levels of phosphorylated Ser129-positive TH+ neurons were statistically significantly higher for cells continually challenged with alpha-synuclein oligomer (“αSyn”) compared to vehicle. Compound 1 interventional treatment significantly reduced levels of phosphorylated Ser129-positive TH+ neurons at doses from 370 nM to 10 μM compared to no treatment.

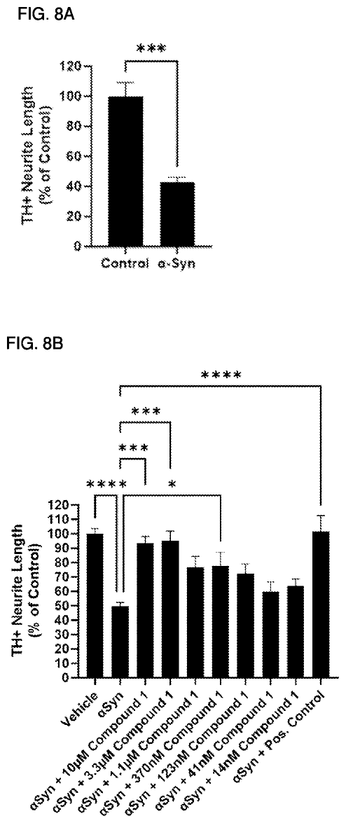

Compound 1 intervention also restored rat dopamine neurite integrity as measured by TH+ neurite length. Cellular challenge with alpha-synuclein oligomer over 2 days afforded lower TH+ neurite length (FIG. 8A). After continued challenge with alpha-synuclein oligomer and adding treatment with Compound 1 over 2 more days, TH+ neurites were longer compared to neurons with no treatment (FIG. 8B). Compound 1 treatment significantly restored TH+ neurite length at doses of 370 nM, 3.3 M, or 10 M compared to no treatment.

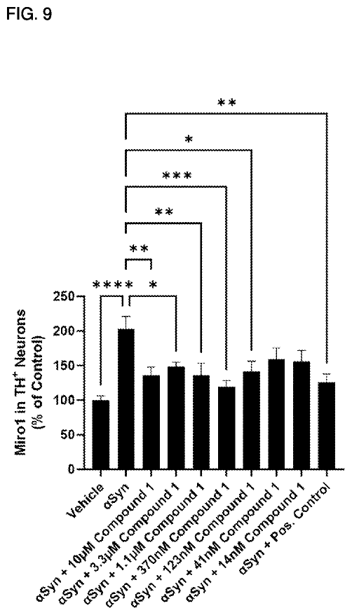

Compound 1 further reduced alpha-Synuclein-induced elevated Miro1 levels in rat midbrain dopamine neuron co-cultures previously challenged with toxic alpha-Synuclein oligomers. At completion of the study (4 days total), Miro1 levels in TH+ neurons were statistically significantly reduced after Compound 1 treatment at doses from 123 nM to 10 M compared to no treatment (FIG. 9).

### Example 11. Compound 9 Treatment in Primary Rat Dopaminergic Neurons after Alpha-Synuclein Oligomer Injury

Interventional treatment with Compound 9 removed pre-existing phospho-serine129 alpha-synuclein pathology in rat midbrain dopamine neuron co-cultures previously challenged with toxic alpha-synuclein oligomers.

Compound 9 was described in Example 2 of PCT application no. PCT/US23/73312 to be ferroptosis inhibitor labeled as Compound 4 having EC₅₀=2.9 nM.

Rat dopaminergic neurons were cultured and challenged with alpha-synuclein oligomer according to the procedure described in Example 10. Subsequent to the challenge, cells were subjected to alpha-synuclein (α-syn) oligomer (500 nM)+various concentrations of Compound 9 (0, 14, 41, 123, 370, 1100, 3300, and 10000 nM) for an additional 48 hours. By condition, N=6 wells were performed and one plate of 96 wells were performed.

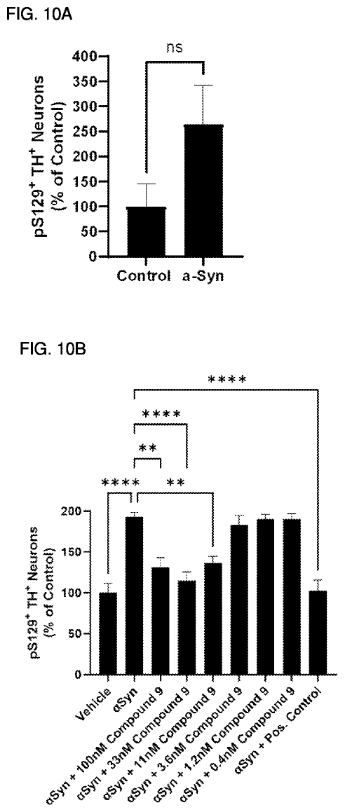

Cellular challenge with alpha-synuclein oligomer over 2 days afforded a trend toward higher levels of phosphorylated Ser129-positive TH+ neurons (FIG. 10A). After continued challenge with alpha-synuclein oligomer and adding treatment with Compound 9 over 2 more days, the levels of phosphorylated Ser129-positive TH+ neurons were reduced compared to no treatment (FIG. 10B). At completion of the study (4 days total), the levels of phosphorylated Ser129-positive TH+ neurons were statistically significantly higher for cells continually challenged with alpha-synuclein oligomer (“αSyn”) compared to vehicle. Compound 9 interventional treatment significantly reduced levels of phosphorylated Ser129-positive TH+ neurons at doses of 11 nM, 33 nM, or 100 nM compared to no treatment.

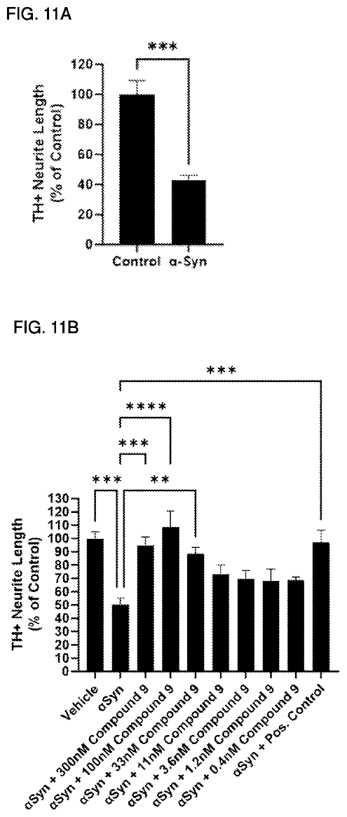

Compound 9 intervention also restored rat dopamine neurite integrity as measured by TH+ neurite length. Cellular challenge with alpha-synuclein oligomer over 2 days afforded lower TH+ neurite length (FIG. 11A). After continued challenge with alpha-synuclein oligomer and adding treatment with Compound 9 over 2 more days, TH+ neurites were longer compared to neurons with no treatment (FIG. 11B). Compound 9 treatment significantly restored TH+ neurite length at doses of 11 nM, 33 nM, or 100 nM compared to no treatment.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of reducing Miro1 and/or phosphorylated alpha-synuclein level in a cell, comprising contacting the cell with an effective amount of a compound having Formula I: 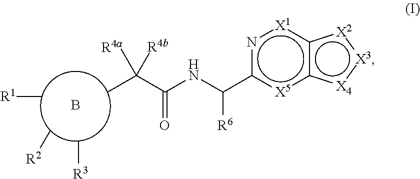
or a pharmaceutically acceptable salt thereof,
wherein
ring B is C₆-C₁₀ aryl or 5- to 10-membered heteroaryl;
R¹, R², and R³ are each independently H, halogen, CN, OR¹¹, NR^{12a}R^{12b}, C₁-C₆ alkyl, C₂-C₆ alkoxyalkyl, C₁-C₆ haloalkyl, C₂-C₆ alkenyl, C₂-C₆ alkynyl, phenyl, 5- to 6-membered heteroaryl, C₃-C₇ cycloalkyl, —(CH₂)ₘ—(C₃-C₇ cycloalkyl), 4- to 7-membered heterocyclyl, or —(CH₂)ₘ-(4- to 7-membered heterocyclyl), wherein the phenyl, heteroaryl, cycloalkyl or heterocyclyl is optionally substituted by 1, 2, or 3 halogen, CN, OR¹¹, NR^{12a}R^{12b}, C₁-C₆ alkyl, C₁-C₆ alkoxy, C₁-C₆ alkylamino, or C₁-C₆ haloalkyl;
R^{4a} and R^{4b} are each independently H or C₁-C₆ alkyl, or R^{4a} and R^{4b} and the carbon atom to which they are attached form a C₃-C₅ cycloalkyl;
R⁶ is H, C₁-C₆ alkyl, or C₁-C₆ haloalkyl;
X¹ and X⁵ are each independently N or CR⁸, provided that at least one of X¹ and X⁵ is CR⁸;
X², X³, and X⁴ are each independently N, NR⁹, O, S, or CR⁹, provided that at least one of X², X³, and X⁴ is N, NR⁹, O, or S;
R⁸ is H, halogen, CN, OR¹, C₁-C₆ alkyl, C₂-C₆ alkoxyalkyl, or C₁-C₆ haloalkyl;
R⁹ is H, C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen, CN, OH, NH₂, C₁-C₆ alkyl, C₁-C₆ alkoxy, C₁-C₆ alkylamino, or C₁-C₆ haloalkyl;
each R¹¹, R^{12a}, and R^{12b} is independently H, C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen or CN; and
subscript m is 1, 2, or 3;
or a compound having Formula II: 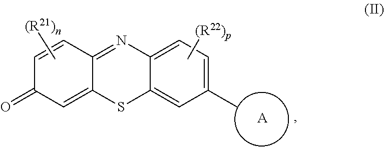
or a pharmaceutically acceptable salt thereof,
wherein
each R²¹ is independently —F, —Cl, —Br, —I, —OR^{a}, —SR^{a}, —NR^{a}R^{b}, —NO₂, —CN, C₁₋₆ alkyl, C₂₋₆ alkenyl, or C₂₋₆ alkynyl, wherein the alkyl, alkenyl, or alkynyl is substituted with 0, 1, 2, or 3 groups independently selected from —F, —Cl, —Br, —I, —OR^{a}, —SR, —NR^{a}R^{b}, oxo, —NO₂, and —CN;
each R²² is independently —F, —Cl, —Br, —I, —OH, —OR^{a}, —SR^{a}, —NR^{a}R^{b}, —CN, C₁₋₆ alkyl, C₂-6 alkenyl, or C₂₋₆ alkynyl, wherein the alkyl, alkenyl, or alkynyl is substituted with 0, 1, 2, or 3 groups independently selected from —F, —Cl, —Br, —I, —OR^{a}, —SR^{a}, —NR^{a}R^{b}, oxo, —NO₂, and —CN;
each R^{a} is independently H or C₁₋₆ alkyl;
each R^{b} is independently H or C₁₋₆ alkyl;
the subscript n is 0, 1, 2 or 3;
the subscript p is 0, 1, 2, or 3; and 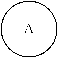
is a nitrogen-containing heterocyclic ring.

2. The method of claim 1, wherein the phosphorylated alpha-synuclein is phosphorylated S129 alpha-synuclein.

3. The method of claim 1 or 2, wherein the reducing Miro1 and/or the phosphorylated alpha-synuclein level is compared to a control Miro1 and/or control phosphorylated alpha-synuclein level in a control cell.

4. The method of any one of claims 1 to 3, wherein the Miro1 and/or the phosphorylated alpha-synuclein level is from about 10% to about 95% lower compared to a control Miro1 and/or control phosphorylated alpha-synuclein level in a control cell after administration of the compound to the cell.

5. The method of any one of claims 1 to 4, wherein the cell is a neuronal cell, a muscle cell, a renal cell, a liver cell, or a skin cell.

6. The method of any one of claims 1 to 5, wherein the compound of Formula I or pharmaceutically acceptable salt thereof is a compound of Formula Ia: 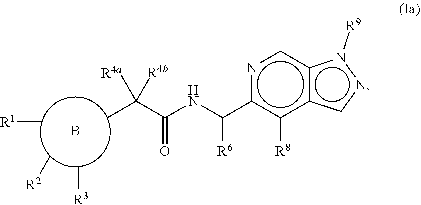
or a pharmaceutically acceptable salt thereof.

7. The method of any one of claims 1 to 6, wherein the compound of Formula I or pharmaceutically acceptable salt thereof is a compound of Formula Ib: 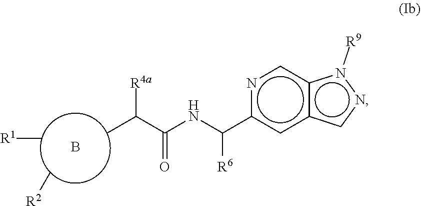
or a pharmaceutically acceptable salt thereof.

8. The method of any one of claims 1 to 7, wherein the compound of Formula I or pharmaceutically acceptable salt thereof is a compound of Formula Ic: 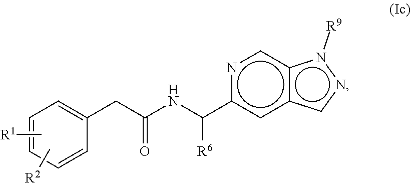
or a pharmaceutically acceptable salt thereof.

9. The method of any one of claims 1 to 8, wherein R¹ and R² are each independently H, C₁-C₃ alkyl, or C₁-C₃ haloalkyl.

10. The method of any one of claims 1 to 9, wherein R⁶ is C₁-C₃ alkyl.

11. The method of any one of claims 1 to 10, wherein R⁹ is C₁-C₃ alkyl, C₂-C₃ alkoxyalkyl, or C₁-C₃ haloalkyl.

12. The method of any one of claims 1 to 11, wherein the compound of Formula I or pharmaceutically acceptable salt thereof is 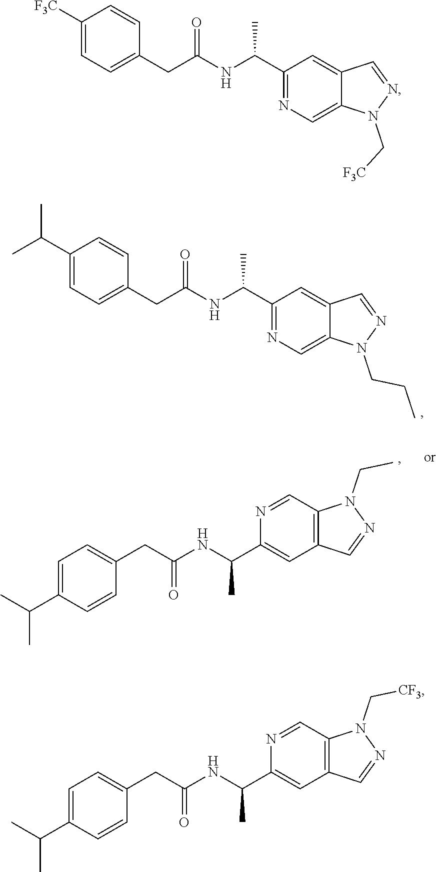
or a pharmaceutically acceptable salt thereof.

13. The method of any one of claims 1 to 5, wherein the compound of Formula II or pharmaceutically acceptable salt thereof is a compound of Formula IIa: 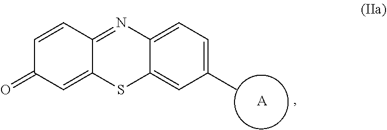
or a pharmaceutically acceptable salt thereof.

14. The method of any one of claims 1 to 5, wherein the compound of Formula II or pharmaceutically acceptable salt thereof s 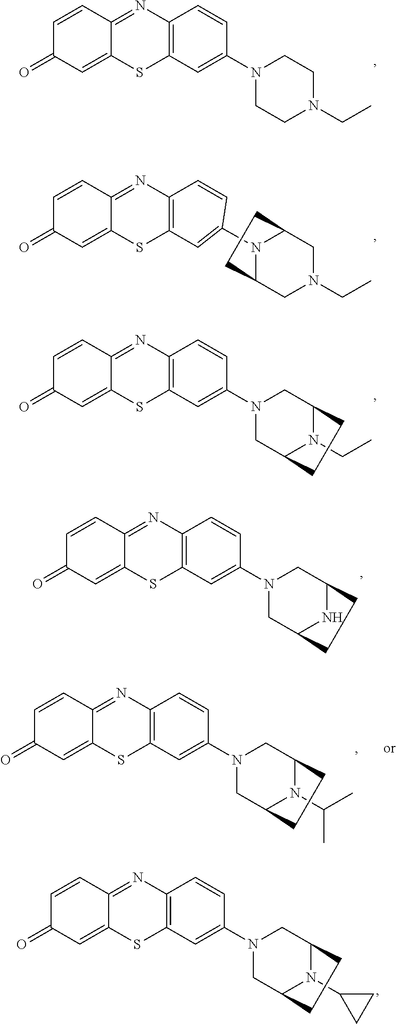 [SMILES: CCN1CCN(c2ccc3nc4ccc(=O)cc-4sc3c2)CC1 ; CCN1C[C@H]2CC[C@@H](C1)N2c1ccc2nc3ccc(=O)cc-3sc2c1 ; CCN1[C@@H]2CC[C@H]1CN(c1ccc3nc4ccc(=O)cc-4sc3c1)C2 ; O=c1ccc2nc3ccc(N4C[C@H]5CC[C@@H](C4)N5)cc3sc-2c1 ; CC(C)N1[C@@H]2CC[C@H]1CN(c1ccc3nc4ccc(=O)cc-4sc3c1)C2 ; O=c1ccc2nc3ccc(N4C[C@H]5CC[C@@H](C4)N5C4CC4)cc3sc-2c1]
or a pharmaceutically acceptable salt thereof.

15. A method of treating a disease or condition characterized by an elevated Miro1 and/or phosphorylated alpha-synuclein level in a subject in need thereof, comprising administering to the subject a therapeutically effective amount of a compound having Formula I: 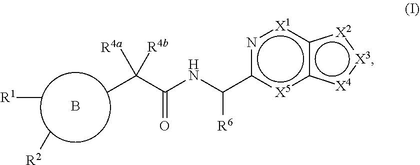
or a pharmaceutically acceptable salt thereof,
wherein
ring B is C₆-C₁₀ aryl or 5- to 10-membered heteroaryl;
R¹, R², and R³ are each independently H, halogen, CN, OR¹¹, NR^{12a}R^{12b}, C₁-C₆ alkyl, C₂-C₆ alkoxyalkyl, C₁-C₆ haloalkyl, C₂-C₆ alkenyl, C₂-C₆ alkynyl, phenyl, 5- to 6-membered heteroaryl, C₃-C₇ cycloalkyl, —(CH₂)ₘ—(C₃-C₇ cycloalkyl), 4- to 7-membered heterocyclyl, or —(CH₂)ₘ-(4- to 7-membered heterocyclyl), wherein the phenyl, heteroaryl, cycloalkyl or heterocyclyl is optionally substituted by 1, 2, or 3 halogen, CN, OR¹¹, NR^{12a}R^{12b}, C₁-C₆ alkyl, C₁-C₆ alkoxy, C₁-C₆ alkylamino, or C₁-C₆ haloalkyl;
R^{4a} and R^{4b} are each independently H or C₁-C₆ alkyl, or R^{4a} and R^{4b} and the carbon atom to which they are attached form a C₃-C₅ cycloalkyl;
R⁶ is H, C₁-C₆ alkyl, or C₁-C₆ haloalkyl;
X¹ and X⁵ are each independently N or CR⁸, provided that at least one of X¹ and X⁵ is CR⁸;
X², X³, and X⁴ are each independently N, NR⁹, O, S, or CR⁹, provided that at least one of X², X³, and X⁴ is N, NR⁹, O, or S;
R⁸ is H, halogen, CN, OR¹¹, C₁-C₆ alkyl, C₂-C₆ alkoxyalkyl, or C₁-C₆ haloalkyl;
R⁹ is H, C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen, CN, OH, NH₂, C₁-C₆ alkyl, C₁-C₆ alkoxy, C₁-C₆ alkylamino, or C₁-C₆ haloalkyl;
each R¹¹, R^{12a}, and R^{12b} is independently H, C₁-C₆ alkyl, C₂-C₆ alkoxyalkyl, C₁-C₆ haloalkyl, C₂-C₆ alkenyl, C₂-C₆ alkynyl, C₃-C₇ cycloalkyl, or 4- to 7-membered heterocyclyl, wherein the cycloalkyl or heterocyclyl is optionally substituted by 1, 2, or 3 halogen or CN; and
subscript m is 1, 2, or 3;
or a compound having Formula II: 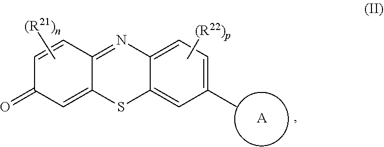
or a pharmaceutically acceptable salt thereof,
wherein
each R²¹ is independently —F, —Cl, —Br, —I, —OR^{a}, —SR^{a}, —NR^{a}R^{b}, —NO₂, —CN, C₁₋₆ alkyl, C₂-6 alkenyl, or C₂₋₆ alkynyl, wherein the alkyl, alkenyl, or alkynyl is substituted with 0, 1, 2, or 3 groups independently selected from —F, —Cl, —Br, —I, —OR^{a}, —SR^{a}, —NR^{a}R^{b}, oxo, —NO₂, and —CN;
each R²² is independently —F, —Cl, —Br, —I, —OH, —OR^{a}, —SR^{a}, —NR^{a}R^{b}, —CN, C₁₋₆alkyl, C₂-6 alkenyl, or C₂₋₆ alkynyl, wherein the alkyl, alkenyl, or alkynyl is substituted with 0, 1, 2, or 3 groups independently selected from —F, —Cl, —Br, —I, —OR^{a}, —SR^{a}, —NR^{a}R^{b}, oxo, —NO₂, and —CN;
each R^{a} is independently H or C₁₋₆ alkyl;
each R^{b} is independently H or C₁₋₆ alkyl;
the subscript n is 0, 1, 2 or 3;
the subscript p is 0, 1, 2, or 3; and 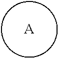
is a nitrogen-containing heterocyclic ring.

16. The method of claim 15, wherein the disease or condition characterized by an elevated Miro1 and/or phosphorylated alpha-synuclein level is a neurodegenerative disease.

17. The method of claim 15 or 16, wherein the disease or condition characterized by an elevated Miro1 and/or phosphorylated alpha-synuclein level is Parkinson's disease.

18. The method of any one of claims 15 to 17, wherein the compound of Formula I or pharmaceutically acceptable salt thereof is a compound of Formula Ia: 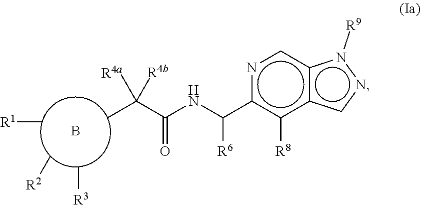
or a pharmaceutically acceptable salt thereof.

19. The method of any one of claims 15 to 18, wherein the compound of Formula I or pharmaceutically acceptable salt thereof is a compound of Formula Ib: 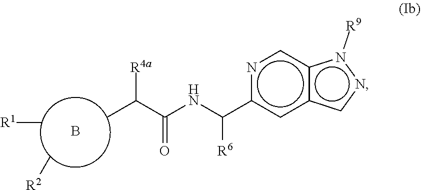
or a pharmaceutically acceptable salt thereof.

20. The method of any one of claims 15 to 19, wherein the compound of Formula I or pharmaceutically acceptable salt thereof is a compound of Formula Ic: 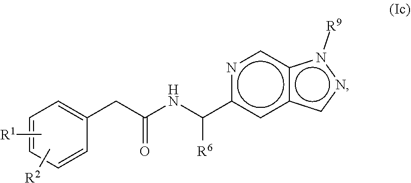
or a pharmaceutically acceptable salt thereof.

21. The method of any one of claims 15 to 20, wherein R¹ and R² are each independently H, C₁-C₃ alkyl, or C₁-C₃ haloalkyl.

22. The method of any one of claims 15 to 21, wherein R⁶ is C₁-C₃ alkyl.

23. The method of any one of claims 15 to 22, wherein R⁹ is C₁-C₃ alkyl, C₂-C₃ alkoxyalkyl, or C₁-C₃ haloalkyl.

24. The method of any one of claims 15 to 23, wherein the compound of Formula I or pharmaceutically acceptable salt thereof is 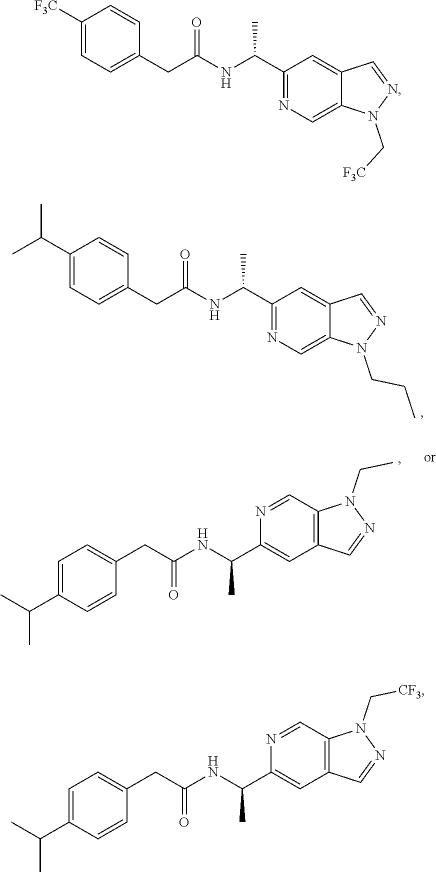
or a pharmaceutically acceptable salt thereof.

25. The method of any one of claims 15 to 17, wherein the compound of Formula II or pharmaceutically acceptable salt thereof is a compound of Formula IIa: 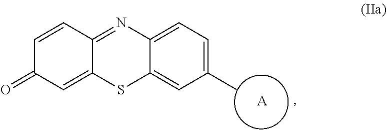
or a pharmaceutically acceptable salt thereof.

26. The method of any one of claims 15 to 17, wherein the compound of Formula II or pharmaceutically acceptable salt thereof is 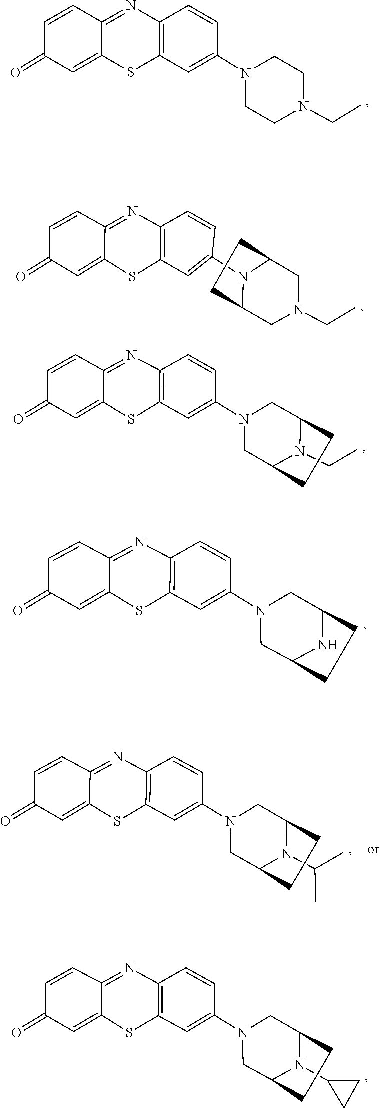 [SMILES: CCN1CCN(c2ccc3nc4ccc(=O)cc-4sc3c2)CC1 ; CCN1C[C@H]2CC[C@@H](C1)N2c1ccc2nc3ccc(=O)cc-3sc2c1 ; CCN1[C@@H]2CC[C@H]1CN(c1ccc3nc4ccc(=O)cc-4sc3c1)C2 ; O=c1ccc2nc3ccc(N4C[C@H]5CC[C@@H](C4)N5)cc3sc-2c1 ; CC(C)N1[C@@H]2CC[C@H]1CN(c1ccc3nc4ccc(=O)cc-4sc3c1)C2 ; O=c1ccc2nc3ccc(N4C[C@H]5CC[C@@H](C4)N5C4CC4)cc3sc-2c1]
or a pharmaceutically acceptable salt thereof.
